(19) United States
(12) Patent Application Publication
(10) Pub. No.: US 2022/0088010 A1
(43) Pub. Date: Mar. 24, 2022

(54) Co-Administration of inhibitors to produce insulin producing gut cells

(71) Applicant: The Trustees of Columbia University in the City of New York, New York, NY (US)

(72) Inventors: Domenico Accili, New York, NY (US); Takumi Kitamoto, Tokyo (JP); Hua V. Lin, Zionsville, IN (US)

(21) Appl. No.: 17/420,955

(22) PCT Filed: Jan. 3, 2020
(86) PCT No.: PCT/US2020/012111

§ 371 Date: Jul. 6, 2021

Related U.S. Application Data
(60) Provisional application No. 62/787,920, filed on Jan. 3, 2019.

(51) Int. Cl. A61K 31/496; A61K 31/55; A61K 31/192; A61K 31/417; A61K 31/4409; A61K 31/551; A61K 31/4184; A61P 3/10

(52) U.S. Cl. CPC A61K 31/496; A61K 31/55; A61K 31/192; A61P 3/10; A61K 31/4409; A61K 31/551; A61K 31/4184; A61K 31/417

(57) ABSTRACT

Methods are described for producing enteroendocrine cells that make and secrete insulin in a subject by co-administering a Foxo1 inhibitor in combination with a Notch inhibitor or ROCK inhibitor, or both. Also described are pharmaceutical compositions comprising a combination of a Foxo1 inhibitor with a Notch inhibitor or ROCK inhibitor, or both. The described methods and compositions may be used to treat a disorder associated with impaired pancreatic function such as diabetes.

### STATEMENT OF GOVERNMENTAL INTEREST

This invention was made with Government support under grants DK057539 and DK58282 awarded by the National Institutes of Health. The Government has certain rights in the invention.

### BACKGROUND

### 1. Field of the Invention

Methods for treating and preventing diabetes.

### 2. Description of the Related Art

Diabetes mellitus is a family of disorders characterized by chronic hyperglycemia and the development of long-term complications. This family of disorders includes type 1 diabetes, type 2 diabetes, gestational diabetes, and other types of diabetes Immune-mediated (type 1) diabetes (or insulin dependent diabetes mellitus, IDDM) is a disease of children and adults for which there currently is no adequate means for cure or prevention. Type 1 diabetes represents approximately 10% of all human diabetes.

Type 1 diabetes is distinct from non-insulin dependent diabetes (NIDDM) in that only the type 1 form involves specific destruction of the insulin producing beta cells of the pancreatic islets of Langerhans; alpha cells (glucagon producing) or delta cells (somatostatin producing) in pancreatic islets are spared. The progressive loss of pancreatic beta cells results in insufficient insulin production and, thus, impaired glucose metabolism with attendant complications. Type 1 diabetes occurs predominantly in genetically predisposed persons. Although there is a major genetic component in the etiology of type 1 diabetes, environmental or non-germline genetic factors also appear to play important roles. Type 1 diabetes affects 1 in 300 people in the U.S. Incidents of type 1 diabetes are rising at the rate of about 3% to 5% per year.

Since 1922, insulin has been the only available therapy for the treatment of type I diabetes and other conditions related to lack of or diminished production of insulin, however, it does not prevent the long-term complications of the disease including damage to blood vessels, nerves, eyes, and kidneys which may affect eyesight, kidney function, heart function and blood pressure and can cause circulatory system complications. This is because insulin treatment cannot replace entirely the missing pancreatic function. Despite decades of research and the advent of pancreatic islet cell transplantation in 1974 and newer claims of success resulting from the Edmonton Protocol for islet cell transplantation, the success of replacing insulin-producing cells has been modest. Difficulties associated with islet or pancreas transplant include obtaining sufficient quantities of tissue and the relatively low rate at which transplanted islets survive and successfully function in the recipient have not yet been overcome. At four years post-transplant, fewer than 10% of patients who have received islet cell transplants remain insulin independent. Additionally, patients require lifelong immune suppression post-transplant, effectively replacing insulin with immune suppressants. And despite new immune suppression protocols, there is an 18% rate per patient of serious side effects.

Therefore, there is a need for additional treatment regimes for the treatment, prevention, and/or reduction in the risk of developing diabetes or other disorders associated with impaired pancreatic function.

Before the embodiments of the present invention are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

### BRIEF DESCRIPTION OF THE DRAWINGS

The present invention is illustrated by way of example, and not by way of limitation, in the figures of the accompanying drawings and in which:

FIG. 1 provides graphs showing the effect of an initial administration of the Notch inhibitor (DBZ) followed by Foxo1 inhibitor (FBT9) administration (24 hrs after DBZ treatment) on body weight and plasma glucose.

FIG. 2 provides micrographs showing the effects of the initial DBZ treatment and subsequent FBT9 sequential treatment on number of Glp1-positive cells: increased in DBZ-only treated animals, no increase in animals treated with a combination of DBZ and FBT9 in duodenum and jejunum.

FIG. 3 provides micrographs showing the effects of the initial DBZ treatment and subsequent FBT9 sequential treatment on the number of Somatostatin-positive cells: increased in DBZ-only treated animals, no increase in animals treated with a combination of DBZ and FBT9.

FIG. 4 provides micrographs showing the effects of the initial DBZ treatment and subsequent FBT9 sequential treatment on number of Serotonin (5HT)-positive cells: increased in DBZ-only treated animals, no increase in animals treated with a combination of DBZ and FBT9.

FIG. 5 provides micrographs showing the effects of the initial DBZ treatment and subsequent FBT9 sequential treatment on number of CCK-positive cells: no increase in either group.

FIG. 6 provides micrographs showing the effects of the initial DBZ treatment and subsequent FBT9 sequential treatment on number of Edu-positive (i.e., replicating) cells: increased in both groups.

FIG. 7 provides micrographs showing the effects of the initial DBZ treatment and subsequent FBT9 sequential treatment on number of insulin-positive cells. The images below the top image are magnified images of the yellow boxes in the top image.

FIG. 8 provides graphs showing the effect of co-treatment of FBT9 and DBZ (DBZ administered in conjunction with first dose of FBT9 followed by sequential administration of FBT9 3 days TID) on body weight and plasma glucose in Foxo1 heterozygous knockouts.

FIG. 9 shows the effect of individual treatment: DBZ alone or FBT9×3 days TID alone neither which produced ins+ cells under this protocol.

FIG. 10 provides micrographs showing the effects of the co-treatment regime described above for FIG. 8 on insulin-positive cells. The number of insulin-positive cells is ˜5-fold higher than the treatment regime described for FIGS. 1-7. The images below the top image are magnified images of the yellow boxes in the top image.

FIG. 11 provides micrographs showing the effects of the co-treatment regime described above for FIG. 8 on 5HT cells.

FIG. 12 provides micrographs showing the effects of the co-treatment regime described above for FIG. 8 on 5HT cells.

FIG. 13 provides micrographs showing the effects of the co-treatment regime described above for FIG. 8 on Glp1 cells: slight increase in the combined treatment.

FIG. 14 provides micrographs showing the effects of the co-treatment regime described above for FIG. 8 on Glp1 cells: slight increase in the combined treatment.

FIG. 15 provides micrographs showing the effects of ROCK inhibitor (“ROCKi”; Y27632) administration in homozygous Foxo1 knockout mice on producing Ins+ cells in the gut. Yellow cells are positive for C-peptide and represent true beta-like cells.

FIG. 16 provides micrographs of Foxo1 knockout mice treated with ROCKi counterstained with Epcam showing that the insulin-positive cells are epithelial.

FIG. 17 provides micrographs showing that the number of insulin-positive cells in Foxo1 knockout mice not treated with ROCKi is significantly lower relative to those treated with ROCKi.

FIG. 18 provides an experiment diagram and series of micrographs showing the effects of FBT10 on gut organoids.

FIG. 19 provides an experiment diagram and series of micrographs showing the effects of a combination of FBT10 and notch signal inhibitor (DBZ) on gut organoids. Also provided are graphs showing the effects on body weight and glucose.

FIG. 20 provides an experiment diagram and series of micrographs showing the effects of FBT10 on gut organoids. Also provided are graphs showing the effects on body weight and glucose.

### SUMMARY

According to one embodiment, disclosed is a method for treating or preventing a disease or disorder in a subject associated with impaired pancreatic function, that includes co-administering to the subject a therapeutically effective amount of a Foxo1 inhibitor and a therapeutically effective amount of a Notch inhibitor or Rock inhibitor, or both. The disease or disorder is selected from the group comprising of diabetes type 1, diabetic type 2, metabolic syndrome, glucose intolerance, hyperglycemia; decreased insulin sensitivity, increased fasting glucose, increased post-prandial glucose and obesity. The therapeutically effective amount is an amount that produces an effect selected from the group consisting of an increase in glucose tolerance, an increase in serum insulin, an increase insulin sensitivity, a decrease in fasting glucose, a decrease in post-prandial glucose, a decrease in weight gain, a decrease in fat mass, an increase in weight loss and the generation of gut Ins+ cells. In a preferred embodiment the agent is administered to the gastrointestinal tract.

Other embodiments are directed to a treating or preventing a disease or disorder in a subject associated with impaired pancreatic function, comprising an effective amount of a Foxo1 inhibitor and a Notch inhibitor or ROCK inhibitor, or both. In some embodiments the effective amount is an amount that produce an effect selected from the group consisting of an increase in glucose tolerance, an increase in serum insulin, an increase insulin sensitivity, a decrease in fasting glucose, a decrease in post-prandial glucose, a decrease in weight gain, a decrease in fat mass, an increase in weight loss and the generation of Gut Ins+ cells.

A method for producing enteroendocrine cells that make and secrete insulin in a subject, comprising co-administering to the subject an effective amount of a Foxo1 inhibitor and an effective amount of a Notch inhibitor or Rock inhibitor, or both. In an embodiment the insulin-producing enteroendocrine cells further produce glucokinase and/or glut2 in response to administration of the agent.

### Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

“Administering” or “administration of a drug or therapeutic pharmaceutical composition to a subject any method known in the art includes both direct administration, including self-administration (including oral administration or intravenous, subcutaneous, intramuscular or intraperitoneal injections, rectal administration by way of suppositories), local administration directly into or onto a target tissue (such as a region of the gut that has Gut Ins−, such as Gut N3 Frog defined below) or administration by any route or method that delivers a therapeutically effective amount of the drug or composition to the cells or tissue to which it is targeted. The term “co-administration” or “co-administering” as used herein refers to the administration of an active agent before, concurrently, or after the administration of another active agent such that the biological effects of either agents overlap. The combination of agents as taught herein can act synergistically to treat or prevent the various diseases, disorders or conditions described herein. Using this approach, one may be able to achieve therapeutic efficacy with lower dosages of each agent, thus reducing the potential for adverse side effects.

An “effective amount” of an agent is an amount that produces the desired effect.

“Enteroendocrine cells” means specialized endocrine cells of the gastrointestinal tract, most of which are daughters of N3 Frog cells that no longer produce Neurogenin 3. Enteroendocrine cells are usually Insulin-negative cells (Gut Ins⁻); and may produce various other hormones such as gastrin, ghrelin, neuropeptide Y, peptide YY 3-36 (PYY 3-36) serotonin, secretin, somatostatin, motilin, cholecystokinin, gastric inhibitory peptide, neurotensin, vasoactive intestinal peptide, glucose-dependent insulinotropic polypeptide (GIP) and glucagon-like peptide-1.

The term “enumerated agent” refers to a Foxo inhibitor, Notch inhibitor and/or ROCK inhibitor.

“An enumerated disease or disorder” means a disease or disorder characterized by impaired pancreatic function including inappropriately low insulin levels, diabetes types 1 and 2, metabolic syndrome, obesity, glucose intolerance, hyperglycemia; decreased insulin sensitivity, increased fasting glucose, increased post-prandial glucose. By inappropriately low insulin levels means insulin levels that are low enough to contribute to at least one symptom of the disease or disorder. Impaired pancreatic function is one in which the pathology is associated with a diminished capacity in a subject for the pancreas to produce and/or secrete insulin and/or an altered capacity (increased or decreased) to secrete pancreatic peptides such as glucagon, pancreatic polypeptide, somatostatin. Disorders associated with impaired pancreatic function include pathologies sometimes referred to as latent autoimmune diabetes of adulthood, pre-diabetes, impaired fasting glucose, impaired glucose tolerance, fasting hyperglycemia, insulin resistant syndrome, and hyperglycemic conditions.

“Foxo1 Gene” means any gene encoding a Foxo1 Protein, including orthologs, and biologically active fragments thereof.

The term “FOXO1 inhibitor” refers to a compound that inhibits completely or partially the activity of a of FOXO1 protein by directly targeting the FOXO1 protein and/or targeting its binding partners, its target genes or the signaling networks controlling FOXO expression. FOXO1 inhibitors or FOXO1 antagonists may include direct inhibitors of FOXO1 activity as well as modulators of FOXO family binding partners (including the androgen receptor, estrogen receptor and smad3), modulators of FOXO family target genes (including p15, p21 and p27) and modulators of the signaling networks controlling FOXO family expression (including Skp2).

“Foxo1 Knock Out Mice” means mice that have been genetically modified to either remove or disrupt Foxo1 expression. Foxo1 Knock Out Mice may be homozygous, where no Foxo1 is expressed or heterozygous where Foxo1 expression is reduced. Not all enteroendocrine cells in the gut of N3 Prog cell-specific Foxo1 knockout mice (hereafter “NKO mice”) make and secrete insulin; some are non-insulin producing (hereafter “Ins−”).

“Foxo1 mRNA” means any mRNA encoding a Foxo1 Protein, including orthologs, and biologically active fragments thereof.

“Gut Ins+ Cells” and “Insulin positive gut cells” means any gut cells that make and secrete insulin. Gut Ins+ cells are descended or converted from Ins− Gut cells. The Gut Ins+ cells have the insulin-positive phenotype (Ins+) so that they express markers of mature beta-cells, and secrete insulin and C-peptide in response to glucose and sulfonylureas. Gut Ins+ Cells arise primarily from N3 Prog and also from gut stem cells. These cells were unexpectedly discovered in NKO (Foxo1 knock out) mice. Unlike pancreatic beta-cells, gut Ins+ cells regenerate following ablation by the beta-cell toxin, streptozotocin, reversing hyperglycemia in mice.

“N3 Enteroendocrine Progenitors” and “N3 Prog” means a subset of insulin-negative gut progenitor cells expressing neurogenin 3 that give rise to Ins⁻ enteroendocrine cells. It has been discovered that N3 Prog in the gut, hereafter “Gut N3 Prog,” have the potential to differentiate into cells that make and secrete insulin (“Gut Ins' Cells”), but this fate is restricted by Foxo1 during development. Pancreatic N3 Prog differentiate into pancreatic insulin-producing cells during fetal development, but it remains unclear whether there is pancreatic N3 Prog after birth or whether pancreatic N3 Prog can differentiate postnatally into pancreatic hormone-producing cells under normal or disordered conditions. It should be noted here that enteroendocrine (gut) and pancreas N3 prog have different features, even though they are commonly referred to as N3 cells.

“Noninsulin-producing gut cells” or “Ins− Gut Cells” broadly means any cells in the gut that are capable of differentiating into an insulin producing gut cell (Gut Ins+ cell), including stem cells, gut progenitor cells, noninsulin producing enteroendocrine cells and N3 Prog.

“Notch inhibitor” refers to an inhibitor of the Notch signaling pathway.

““ROCK inhibitor” or “ROCKi” refers to a compound that reduces the biological activity of Rho Kinase (ROCK; either ROCK 1 or ROCK 2, e.g. Genbank Accession No. NM-005406 or e.g. Genbank Accession No. NM_004850); or that reduces the expression of an mRNA encoding a ROCK polypeptide; or that reduces the expression of a ROCK polypeptide.

“Pathology associated with impaired pancreatic function” or pancreatic malfunction is one in which the pathology is associated with a diminished capacity in a subject for the pancreas to produce and/or secrete one or more pancreatic hormones including insulin and/or pancreatic peptides such as glucagon, pancreatic polypeptide, or somatostatin. Pathologies that are associated with impaired pancreatic function include type 1 diabetes, and type 2 diabetes. Other pathologies include those sometimes referred to as latent autoimmune diabetes of adulthood, pre-diabetes, impaired fasting glucose, impaired glucose tolerance, fasting hyperglycemia, insulin resistant syndrome, and hyperglycemic conditions. Other pathologies include gestational diabetes, maturity onset diabetes of the young (MODY), and insulin dependence secondary to pancreatectomy.

By “pharmaceutically acceptable”, it is meant the carrier, diluent or excipient must be compatible with the other ingredients of the formulation and not deleterious to the recipient thereof.

“Preventing a disease” includes, but is not limited to, preventing the disease from occurring in a subject that may be predisposed to the disease (or disorder), but has not yet been diagnosed as having the disease; inhibiting the disease, for example, arresting the development of the disease; relieving the disease, for example by causing its regression; relieving the condition caused by the disease, for example by reducing its symptoms, and/or delaying disease onset. An example is reducing blood glucose levels in a hyperglycemic subject, and/or maintaining acceptable control of blood glucose levels in the subject. Such treatment, prevention, symptoms and/or conditions can be determined by one skilled in the art and are described in standard textbooks.

A “prophylactically effective amount” of a drug is an amount of a drug that, when administered to a subject, will have the intended prophylactic effect, e.g., preventing or delaying the onset (or reoccurrence) of the disease or symptoms, or reducing the likelihood of the onset (or reoccurrence) of the disease or symptoms. The full prophylactic effect does not necessarily occur by administration of one dose and may occur only after administration of a series of doses. Thus, a prophylactically effective amount may be administered in one or more administrations. For diabetes, a therapeutically effective amount can also be an amount that increases insulin secretion, increases insulin sensitivity, increases glucose tolerance, or decreases weight gain, weight loss, or fat mass.

“Reduction” of a symptom(s) means decreasing of the severity or frequency of the symptom(s), or elimination of the symptom(s).

“Stem Cells” means undifferentiated, cells that can self-renew for unlimited divisions and differentiate into multiple cell types. “Progenitor cells” in the gut means cells descended from stem cells that are multipotent, but self-renewal property is limited.

By significantly lower in the context of reducing expression or biological activity of a Foxo1 protein is meant lowering the level of Foxo1 protein enough so that the enteroendocrine or other non-insulin-producing cell acquires an Ins+ phenotype, including expressing insulin.

Significantly higher than the level in the control in an assay means detectable by commonly employed assays (elisa or ria), whereas in the control population insulin cannot be detected by such assays. Significantly decreased levels of Foxo1 protein expression is intended as a decrease that is greater than 50% of the control values (note: we know that up to 50% decrease nothing happens, so the decrease has to be greater than 50%).

In the context of determining the level of insulin expression in the control and the test population after contacting with an agent that causes the test population to become insulin-producing cells, significantly higher means any reliably detectable level of insulin since untreated cells are noninsulin-producing. A person of skill in the art of screening assays can define significantly higher or significantly lower depending on the assay.

A “subject” or “patient” is a mammal, typically a human, but optionally a mammalian animal of veterinary importance, including but not limited to horses, cattle, sheep, dogs, and cats.

A “therapeutically effective amount” of an active agent or pharmaceutical composition is an amount that achieves the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

“Treating” a disease, disorder or condition in a patient refers to taking steps to obtain beneficial or desired results, including clinical results. For purposes of this disclosure, beneficial or desired clinical results include, but are not limited to alleviation or amelioration of one or more symptoms of the disease; diminishing the extent of disease; delaying or slowing disease progression; amelioration and palliation or stabilization of the disease state.

Where the disease is diabetes type 1, symptoms include frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections. These symptoms are associated with characteristic clinical laboratory findings that include hyperglycemia (excessively elevated sugar concentrations in the blood, i.e. ≥125 mg/dl), loss of glycemic control (i.e., frequent and excessive swings of blood sugar levels above and below the physiological range, generally maintained between 70-110 mg/dl), fluctuations in postprandial blood glucose, fluctuations in blood glucagon, fluctuations in blood triglycerides and include reduction in rate of or diminution of or improved outcomes of conditions that are accelerated by and/or occur because of or more frequently with diabetes including microvascular and microvascular disease inclusive but not limited to cerebrovascular impairment with or without, stroke, angina, coronary heart disease, myocardial infarction, peripheral vascular disease, nephropathy, kidney impairment, increased proteinuria, retinopathy, neovascularization of vessels in the retina, neuropathy including central, autonomic and peripheral neuropathy that may lead to loss of sensation of extremities and amputation and/or from neuropathy or diminished vascular flow, skin conditions including but not limited to diabetic dermopathy, Necrobiosis Lipoidica Diabeticorum, bullosis diabeticorum, scleroderma diabeticorum, granuloma annulare, bacterial skin infections, but limited to Staphylococcus, which can result in deeper infections, and gastoparesis (abnormal emptying of the stomach). Type 1 diabetes may be diagnosed by methods well known to one of ordinary skill in the art. For example, commonly, diabetics have a plasma of fasting blood glucose result of greater than 126 mg/dL of glucose. Prediabetes is commonly diagnosed in patients with a blood glucose level between 100 and 125 mg/dL of glucose. Other symptoms may also be used to diagnose diabetes, related diseases and conditions, and diseases and conditions affected by diminished pancreatic function.

Where the disease is type 2 diabetes, symptoms include: a fasting plasma glucose concentration (FPG) that is ≥7.0 mmol/L (126 mg/dl), or the post challenge plasma glucose concentration is ≥11.1 mmol/L (200 mg/dl), performed as described by the World Health Organization (Definition, Diagnosis and Classification of Diabetes Mellitus and its Complications. Part 1: Diagnosis and Classification of Diabetes Mellitus. WHO/NCD/NCS/99.2. Geneva; 1999), using a glucose load containing the equivalent of 75 g anhydrous glucose dissolved in water, or HbA1c values of ≥6.5%, or symptoms of diabetes and a casual plasma glucose ≥200 mg/dl (11.1 mmol/L). These criteria are described in the Global IDF/ISPAD Guideline for Diabetes in Childhood and Adolescence (International Diabetes Federation, ISBN 2-930229-72-1). Depending on the obtained test results, subjects can be diagnosed as being normal, pre-diabetes or diabetes subjects. Pre-diabetes precedes the onset of type 2 diabetes. Generally, subjects who have pre-diabetes have fasting blood glucose levels that are higher than normal, but not yet high enough to be classified as diabetes. Pre-diabetes greatly increases the risk for diabetes. Type 2 diabetes is a progressive disease that over time if not controlled leads a need for insulin administration, i.e., insulin dependence.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### DETAILED DESCRIPTION

Embodiments of the present disclosure build on the discovery that inhibition of Foxo1 in gut cells caused a production of Insulin-positive enteroendocrine cells (Gut Ins' Cells) that make and secrete biologically active insulin and C-peptide, as well as other pancreatic hormones and transcription factors. Importantly the Gut Ins' cells secreted insulin in a dose-dependent manner in response to glucose. The ability of Gut Ins' cells to secrete insulin in direct proportion to the concentrations of glucose in the environment is a key feature of healthy insulin-producing cells in the pancreas that thus far no other group has been able to replicate. In addition, insulin release can be blocked by the Potassium channel opener, diazoxide, effectively providing a safety mechanism to prevent unwanted, excessive insulin release. It has now been discovered that co-administration of Foxo1 with either a Notch inhibitor or ROCK inhibitor, or both further potentiates the ability to generate Ins+ Gut Cells. It has also been determined that the timing of the administration of inhibitors can increase the effect. In a particular embodiment, a Foxo1 inhibitor and Notch inhibitor are initially administered contemporaneously followed by a sequential administration of the Foxo1 inhibitor to treat diabetes in a subject. In another specific embodiment, a Foxo1 inhibitor and a ROCK inhibitor are co-administered to treat diabetes in a subject.

Based at least in part on these discoveries, certain embodiments of the invention are directed to methods for producing mammalian Gut Ins' cells by contacting Gut Ins⁻ cells with a combination of agents that causes the cells to become Gut Ins' cells. In a specific embodiment, the combination of agents pertains to a Foxo1 inhibitor and Notch inhibitor and/or ROCKi. The Gut Ins− cells can be contacted with the agent in situ in the animal, or enriched populations of Gut Ins⁻ can be isolated from the gut, or intestinal explants in culture can be used. Certain other embodiments are directed to the isolated Gut Ins' cells themselves, and to tissue explants that include Gut Ins' cells, preferably intestinal tissue but artificial tissues are also included. Additional methods include the generation of Ins+ cells from cells that have been reprogrammed in vitro to become gut ins− cells. In other words, gut ins− cells that have been obtained indirectly through manipulation of other cell types. These methods and others known in the art can be used in the embodiments of the invention. Maehr R, et al., 2009 Sep. 15; 106(37):15768-73. Epub 2009 Aug. 31, Generation of pluripotent stem cells from patients with type 1 diabetes.

Efficacy of the methods of treatment described herein can be monitored by determining whether the methods ameliorate any of the symptoms of the disease being treated. Alternatively, one can monitor the level of serum insulin or C-peptide (a byproduct of insulin secretion and an index of functional Ins+ cells), which levels should increase in response to therapy. Alternatively, efficacy can be measured by monitoring glycemia, glucose tolerance, fat mass, weight gain, ketone bodies or other indicia of the enumerated disease or disorder in the subject being treated.

In addition to reduced insulin secretion, impaired pancreatic function includes an altered capacity to produce and/or secrete one or more pancreatic hormones including one or more pancreatic peptides such as glucagon, pancreatic polypeptide, somatostatin, IAPP (islet amyloid polypeptide), amylin or ghrelin. Well known pathologies that are associated with impaired pancreatic function include type 1 diabetes, and type 2 diabetes. Other pathologies include those sometimes referred to as latent autoimmune diabetes of adulthood, pre-diabetes, impaired fasting glucose, impaired glucose tolerance, fasting hyperglycemia, insulin resistant syndrome, and hyperglycemic conditions. All of these come within the meaning of treating and preventing diabetes.

It has also been discovered that insulin secretion by Gut Ins' cells can be shut off using the drug diazoxide, which is an important safety measure for controlling any unwanted insulin-production in an animal that has been induced to make Gut Ins' cells or that has been treated by administering Gut Ins' cells as a therapeutic method for treating a disease associated with low insulin production or impaired pancreatic function.

Therefore, certain embodiments of the invention are directed to methods for treating or preventing type 1 or type 2 diabetes, or another of the enumerated diseases or disorders as defined herein that are associated with inappropriately low insulin or impaired pancreatic function in an animal by co-administering a therapeutically effective amount of Foxo1 inhibitor with a Notch inhibitor and/or ROCK inhibitor to produce Gut Ins' cells. In some other embodiments these disorders are treated or prevented by administering to a subject in need of such treatment a therapeutically effective amount of Gut Ins' cells, preferably autologous or partial autologous cells.

### Enumerated Agents

Foxo

The term “FOXO1 inhibitor” refers to a compound that inhibits completely or partially the activity of a of FOXO1 protein by directly targeting the FOXO1 protein and/or targeting its binding partners, its target genes or the signaling networks controlling FOXO expression. Foxo1 inhibitors may also target the protein for degradation, prevent its nuclear import, interfere with its binding to DNA or to other effectors of the transcriptional process that result in the inability to regulate gene expression. FOXO1 inhibitors or FOXO1 antagonists may include direct inhibitors of FOXO1 activity as well as modulators of FOXO family binding partners (including the androgen receptor, estrogen receptor and smad3), modulators of FOXO family target genes (including p15, p21 and p27) and modulators of the signaling networks controlling FOXO family expression (including Skp2). Thus, the term “FOXO1 inhibitor” is intended to include, but is not limited to, molecules which neutralize the effect of FOXO1, in particular its function as a transcription factor. FOXO binding partners include: androgen receptor, β-catenin, constitutive androstane receptor, Cs1, C/EBPα, C/EPBβ, estrogen receptor, FoxG1, FSH receptor, HNF4, HOXA5, HOXA10, myocardin, PGC-1α, PPARα, PPARγ, PregnaneX receptor, progesterone receptor, retinoic acid receptor, RUNX3, smad3, smad4, STATS, thyroid hormone receptor (van der Vos and Coffer, 2008, Oncogene 27:2289-2299). FOXO family target genes include: BIM-1, bNIP3, Bcl-6, FasL, Trail (cell death), catalase, MnSOD, PA26 (detoxification); GADD45, DDB1 (DNA repair), p27KIP1, GADD45, p21CIP1, p130, Cyclin G2 (cell cycle arrest), Glucokinase, G6Pase, PEPCK (glucose metabolism), NPY, AgRP (energy homeostasis), BTG-1, p21CIP1 (differentiation), atrogin-1 (atrophy) (Greer and Brunet, 2005, Oncogene, 24(50):7410-25). Modulators of signaling networks controlling FOXO expression include Skp2 (Huang and Tindall, 2007, Journal of Cell Science 120:2479-248). Hausler et al, Nat Commun. 2014 Oct. 13; 5:5190 sets forth a number of other Foxo targets.

FOXO1 inhibitors inhibit or reduce biological activity or expression of Foxo1. Foxo1 inhibitors may include small molecules, peptides, peptidomimetics, agents that promote protein degradation (e.g., by targeting it to the proteasome), chimeric proteins, natural or unnatural proteins, nucleic acids or nucleic acid derived polymers such as DNA and RNA aptamers, siRNAs (small interfering RNAs), shRNAs (short hairpin RNAs), anti-sense nucleic acid, microRNA (miRNA), or complementary DNA (cDNA), PNAs (Peptide Nucleic Acids), or LNAs (Locked Nucleic Acids), antibody antagonists such as neutralizing anti-FOXO1 antibodies, or expression vectors driving the expression of such FOXO1 inhibitors.

Small molecule inhibitors of Foxo1 include, but are not limited to 5-amino-7-(cyclohexylamino)-1-ethyl-6-fluoro-4-oxo1,4-dihydroquinoline-3-carboxylic acid (AS1842856), 1-cyclopentyl-6-fluoro-4-oxo-7-(tetrahydro-2H-pyran-3-ylamino)-1,4-dihydroquinoline-3-carboxylic acid (AS1841674), 7-(cyclohexylamino)-6-fluoro-4-oxo-1-(prop-1-en-2-yl)-1,4-dihydroquinoline-3-carboxylic acid (AS1838489), 7-(cyclohexylamino)-6-fluoro1-(3-fluoroprop-1-en-2-yl)-4-oxo-1,4-dihydroquinoline-3-carboxylic acid (AS1837976), 7-(cyclohexylamino)-1-(cyclopent-3-en-1-yl)-6-fluoro-4-oxo-1,4-dihydroquinoline-3-carboxylic acid (AS1805469), 7-(cyclohexylamino)-6-fluoro-5-methyl-4-oxo-1-(pentan-3-yl)-1,4-dihydroquinoline-3-carboxylic acid (AS1846102) (Nagashima et al., 2010, Molecular Pharmacology 78: 961-970), 2-Cyclopentyl-N-[2,4-dichloro-3-(isoquinolin-5-yloxymethyl)phenyl] N-methylacetamide (AS1708727) (Tanaka et al., European Journal of Pharmacology 645: 185-191), 2-(2-(methylamino)pyrimidin-4-yl)-1,5,6,7-tetrahydro-4H-pyrrolo[3,2-c]pyridine-4-one (compound 8), N-(3-(1H-benzo[d]imidazole-2-yl)-1H-pyrazol-5-yl)-3-chloro-4-methoxybenzamide (compound 9), N-(3-(1H-benzo[d]imidazol-2-yl)-1H-pyrazol-5-yl)-4-(4-methylpiperazin-1-yl)benzamide (compound 10), (2-chloro-4-((4-(1-isopropyl-2-methyl-1H-imidazol-5-yl)pyrimidin-2-yl)amino)phenyl)(1,4-oxazepan-4-yl)methanone (compound 11), 2-(2-((4-((4-(1-isopropyl-2-methyl-1H-imidazol-5-yl)pyrimidin-2-yl)amino)phenyl)sulfonyl)ethoxy)ethan-1-ol (compound 12), and 7-(3-methoxypyridin-4-yl)pyrrolo[1,2-a]pyrazin-1(2H)-one (compound 13) (Langlet et al., 2017, Cell 171, 824-835).

Examples of siRNAs or shRNAs targeting FOXO1 include siRNA #6242 (Alikhani et al., 2005, J. Biol. Chem. 280: 12096-12102) and examples of antibodies directed against FOXO1 include antibody #9454 (Kanao et al., 2012, PloS ONE 7(2), e30958), antibodies H128 and ac11350 (Liu et al., PLoS ONE 8(2), e58913). FOXO1 inhibitors also include molecules which inhibit the proper nuclear localization of FOXO1 such as, for instance, proteins encoded by any one of the genes selected from the group consisting of: serum/glucocorticoid regulated kinase (Accession No.: BC016616), FK506 binding protein 8 (Acc. No.: BC003739), apolipoprotein A-V (Acc. No.: BC011198), stratifin (Acc. No.: BC000995), translocation protein 1 (Acc. No.: BC012035), eukaryotic translation elongation factor 1 alpha 1 (Acc. No.: BC010735), lymphocyte cytosolic protein 2 (Acc. No.: BC016618), sulphide quinone reductase-like (Acc. No.: BC011153), serum/glucocorticoid regulated kinase-like (Acc. No.: BC015326), tyrosine 3-monooxygenase/tryptophan 5-monooxygenase activation protein, zeta polypeptide (Acc. No.: BC003623), tyrosine 3-monooxygenase/tryptophan 5-monooxygenase activation protein, gamma polypeptide (Acc. No.: BC020963) as described in Table 2 of US 2009/0156523.

FOXO1 inhibitors may also include dominant-negative mutants of FOXO1. Examples of such mutants are described in Nakae et al., J Clin Invest, 2001 108(9):1359-1367. A specific example of a FOXO1 dominant-negative mutant is 4256 mutant Foxo1. The dominant-negative FOXO1 mutant may be administered in protein form or may be expressed in vivo via an expression vector.

FOXO Proteins

The defining feature of Foxo proteins is the forkhead box or motif, a DNA-binding domain having about 80 to 100 amino acids that and is made up of three helices and two characteristic large loops, or “wings.” Following a standardized nomenclature for these proteins, all uppercase letters are used for human (e.g., FOXO1), and only the first letter is capitalized for mouse (e.g., Foxo1). The FOXO1 gene identified in Genbank NM_002015.3) (previously also FOXO1; FKH1; FKHR; and FOXO1A) is the most abundant FOXO isoform in insulin-responsive tissues such as hepatic, adipose, and pancreatic cells. FOXO4 (aka AFX; AFX1; MLLT7; MGC120490; FOXO4) is set forth in Genbank NM_005938.3); FOXO3 (aka, FOXO2; AF6q21; FKHRL1; FOXO3A; FKHRL1P2; MGC12739; MGC31925; DKFZp781A0677); is set forth in Genbank NM_001455.3. All are incorporated herein by reference. Persons of skill in the art will be able to construct appropriate antisense nucleotides and siRNA using methods known in the art based on this sequence.

The significant homology between the genes encoding the various FOXO proteins and the proteins themselves in animals, including humans and mice, means that shRNA, SiRNA and antisense RNA or DNA that target FOXO1 mRNA or the gene may also be sufficiently complementary to FOXO3 and FOXO4, to reduce their expression, Similarly, siRNA and antisense designed to target FOXO4 or FOXO3 may be sufficiently complementary to FOXO1 to reduce its expression. Because the experiments were conducted on mice, the lower case nomenclature was used throughout, however, as used herein “Foxo” means any Foxo protein, gene or mRNA from any species. For the purpose of the methods and compositions of the invention, “Foxo proteins” includes orthologs (analogs in different species) like Foxo1 and biologically active fragments thereof. In certain embodiments the desired Gut Ins' phenotype is produced by reducing the expression or activity of one or more Foxo proteins, for example Foxo1.

Because of the sequence homology, antisense or siRNA made against mouse Foxo1 might be used in other animals including humans, and vice versa. All of the gene IDs and accession numbers and the corresponding nucleotides encoding Foxo proteins, genes, mRNA and cDNA are hereby expressly incorporated by reference in their entirety.

TABLE 1
GENE ID NUMBERS FOR FOXO GENES AND mRNA

| | | | | |
|---|---|---|---|---|
| Gene symbol | Gene Symbol: | Gene Symbol: | Gene Symbol: | Gene Symbol: |
| FOXO1 | FOXO1 | Foxo1 | Foxo3 | FOXO3 |
| Alternate Symbols: | Alternate | Alternate | Alternate | Alternate Symbols: |
| Afxh, FKHR, Fkhr1, | Symbols: | Symbols: | Symbols: | AF6q21, |
| Foxo1a | FKH1, FKHR, | Fkhr, Foxo1a | 1110048B16Rik, | DKFZp781A0677, |
| Organism: | FOXO1A | Organism: | 2010203A17Rik, | FKHRL1, |
| Mouse | Organism: | Rat | C76856, FKHRL1, | FKHRL1P2, FOXO2, |
| Gene Id: | Human | Gene Id: | Fkhr2, Foxo3a | FOXO3A, MGC12739, |
| 56458 | Gene Id: | 84482 | Organism: | MGC31925 |
| Gene Name: | 2308 | Gene Name: | Mouse | Organism: |
| forkhead box O1 | Gene Name: | forkhead box | Gene Id: | Human |
| Accession | forkhead box O1 | O1 | 56484 | Gene Id: |
| Numbers: | Accession | Accession | Gene Name: | 2309 |
| NM_019739 | Numbers: | Numbers: | forkhead box O3 | Gene Name: |
| | NM 002015 | XM 001056726; | Accession | forkhead box O3 |
| | | XM_342244 | Numbers: | Accession |
| | | | NM_019740 | Numbers: |
| | | | | NM_001455; |
| | | | | NM_201559 |
| Gene Symbol: | Gene Symbol: | Gene Symbol: | Gene Symbol: | |
| FOXO4 | Foxo4 | Foxo4 | Foxo3 | |
| Alternate Symbols: | Alternate | Alternate | Alternate | |
| AFX, AFX1, | Symbols: | Symbols: | Symbols: | |
| MGC120490, MLLT7 | afx, Afxh, Foxo4, | LOC302415, | Fkhrl1, Foxo3a | |
| Organism: | Afxh, | RGD1561201 | Organism: | |
| Human | MGC117660, | Organism: | Rat | |
| Gene Id: | Mllt7 | Rat | Gene Id: | |
| 4303 | Organism: | Gene Id: | 294515 | |
| Gene Name: | mouse | 302415 | Gene Name: | |
| forkhead box O4 | Gene Id: | Gene Name: | forkhead box O3 | |
| Accession | 54601 | forkhead box | Accession | |
| Numbers: | Gene Name: | O4 | Numbers: | |
| NM_005938 | forkhead box O4 | Accession | NM_001106395 | |
| | Accession | Number | | |
| | Number | NM_001106943.1 | | |
| | NM_019739.3 | | | |

Homo sapiens forkhead box O1 (FOXO1), mRNA

### NCBI Reference Sequence: NM_002015.3

Mus musculus forkhead box O1 (Foxo1), mRNA

### NCBI Reference Sequence: NM_019739.3

Rattus norvegicus forkhead box O1 (Foxo1), mRNA

### NCBI Reference Sequence: NM_001191846.1

Homo sapiens forkhead box 03 (FOXO3), transcript variant 1, mRNA

### NCBI Reference Sequence: NM_001455.3

Homo sapiens forkhead box 03 (FOXO3), transcript variant 2, mRNA

### NCBI Reference Sequence: NM_201559.2

Mus musculus forkhead box 03 (Foxo3), mRNA

### NCBI Reference Sequence: NM_019740.2

Rattus norvegicus forkhead box 03 (Foxo3), mRNA

### NCBI Reference Sequence: NM_001106395.1

Homo sapiens forkhead box 04 (FOXO4), transcript variant 2, mRNA

### NCBI Reference Sequence: NM_001170931.1

Homo sapiens forkhead box 04 (FOXO4), transcript variant 1, mRNA

### NCBI Reference Sequence: NM_005938.3

Rattus norvegicus forkhead box 04 (Foxo4), mRNA

### NCBI Reference Sequence: NM_001106943.

Mus musculus forkhead box 04 (Foxo4), mRNA

### NCBI Reference Sequence: NM_018789.2

Genomic RefSeqGene, FOXO1 human, NG_023244.1.
Foxo1 Mus musculus strain C57BL/6J chromosome 3, MGSCv37 C57BL/6J, NC_000069.5.

### Foxo1 Rat, NC_005101.2, NW_047625.2.

FOXO3 human, NC_000006.11.
Foxo3 mouse, NC_000076.5.

### Foxo3 Rat, NC_005119.2.

FOXO4 human, NC_000023.10.
Foxo4 mouse, NC_000086.6.
Foxo4 rat, NC_005120.2.
forkhead box O1 [Mus musculus]

### GenBank: EDL35224.1

Forkhead protein FKHR1 [Mouse]

### Swiss-Prot: Q9WVH5

forkhead box protein O1 [Homo sapiens]

### NCBI Reference Sequence: NP_002006.2

forkhead box protein O1 [Rattus norvegicus]

### NCBI Reference Sequence: NP_001178775.1

forkhead box protein O3 [Homo sapiens]

### NCBI Reference Sequence: NP_963853.1

forkhead box protein O3 [Homo sapiens]

### NCBI Reference Sequence: NP_001446.1

forkhead box protein O3 [Rattus norvegicus]

### NCBI Reference Sequence: NP_001099865.1

forkhead box protein O3 [Mus musculus]

### NCBI Reference Sequence: NP_062714.1

forkhead box protein O4 [Rattus norvegicus]

### NCBI Reference Sequence: NP_001100413.1

forkhead box protein O4 isoform 2 [Homo sapiens]

### NCBI Reference Sequence: NP_001164402.1

forkhead box protein O4 isoform 1 [Homo sapiens]

### NCBI Reference Sequence: NP_005929.2

forkhead box protein O4 [Mus musculus]

### NCBI Reference Sequence: NP_061259.1

### Notch Inhibitors

The Notch signaling pathway has been identified as playing an important role in many diverse biological functions, including differentiation, and cellular proliferation (see U.S. Pat. No. 6,703,221). This pathway is activated by four different transmembrane receptor subtypes (designated as Notchl-Notch4) that rely on regulated proteolysis. Expression patterns and functions of Notch depend on cell type and context. Following ligand binding, the receptor undergoes sequential cleavage by metalloproteases of the ADAM family (Bru, et al., Mol. Cell 5:207-216 (2000); Mumm, et al., Mol. Cell 5:197-206 (2000)) and the presenilin-dependent gamma-secretase (Selkoe, et al., Annu. Rev. Neurosci. 26:565-97 (2003); De Strooper, et al., Nature 398:518-522 (1999)). The final proteolytic cleavage step permits the intracellular domain of the Notch receptor to translocate to the cell nucleus where it interacts with transcription factors to induce target gene expression.

In the cell nucleus, the Notch intracellular domain undergoes ubiquitination. Proteolytic processing of the Notch precursor protein by furin-protease and its trafficking to the cell membrane also determine turnover and availability of receptors, and, in turn, activation of this signaling pathway. Altered glycosylation of the Notch extracellular domain by Fringe protein family members may also modify efficiency of ligand binding.

The Notch pathway contributes to biological processes during development and to disease mechanisms in adults (Bray, et al., Nat. Rev. Mol. Cell. Biol. 7:678-689 (2006); Artavanis-Tsakonas, et al., Science 284:770-776 (1999)). Direct cell-to-cell contract via the binding of a ligand to a Notch receptor, both of which are expressed on the cell surface, triggers downstream responses (Thurston, et al., Nat. Rev. Cancer 7:327-331 (2007)).

A Notch inhibitor prevents or inhibits, in part or in whole, the activity of components of the Notch pathway. In one example, a component of the Notch pathway is a Notch protein, which includes notch or other protein involved in the notch signaling pathway. Notch pathway inhibitors are known in the art. In some embodiments, a Notch inhibitor is a gamma secretase inhibitor (GSI). Gamma secretase is a multi-subunit protease complex that cleaves Notch. This cleavage releases Notch from the cell membrane, allowing Notch to enter the nucleus and modify gene expression.

Notch inhibitors that can be provided as a part of a treatment can include small molecules, peptides, peptidomimetics, chimeric proteins, natural or unnatural proteins, nucleic acids or nucleic acid derived polymers such as DNA and RNA aptamers, siRNAs (small interfering RNAs), shRNAs (short hairpin RNAs), anti-sense nucleic acid, microRNA (miRNA), or complementary DNA (cDNA), PNAs (Peptide Nucleic Acids), or LNAs (Locked Nucleic Acids), fusion proteins with Notch antagonizing activities, antibody antagonists such as neutralizing anti-Notch antibodies, or expression vectors driving the expression of such Notch inhibitors.

Small molecule Notch inhibitors include, but are not limited to, DAPT; LY411575; MDL-28170; R04929097; L-685458 ((5S)-(t-Butoxycarbonylamino)-6-phenyl-(4R)hydroxy-(2R)benzylhexanoyl)-L-leu-L-phe-amide); BMS-708163 (Avagacestat); BMS-299897 (2-R1R)-1-[[(4-Chlorophenyl)sulfonyl](2,5-difluorophenyl)amino]ethyl-5-fluorobenzenebutanoic acid); M-0752; YO-01027; MDL28170 (Sigma); LY41 1575 (N-2((2S)-2-(3,5-difluorophenyl)-2-hydroxyethanoyl)-N1-((7S)-5-methyl-6-oxo-6,7-dihydro-5H-dibenzo[b,d]azepin-7-yl)-1-alaninamide); ELN-46719 (2-hydroxy-valeric acid amide analog of LY41 1575; PF-03084014 ((S)-2-((S)-5,7-difluoro-1,2,3,4-tetrahydronaphthalen-3-ylamino)-N-(1-(2-methyl-1-(neopentylamino)propan-2-yl)-1H-imidazol-4-yl)pentanamide); Compound E ((2S)-2-[[(3,5-Diflurophenyl)acetyl]amino]-N-[(3S)-1-methyl-2-oxo-5-phenyl-2,3-dihydro-1H-1,4-benzodiazepin-3-yl]propanamide; and Semagacestat (LY450139); (2S)-2-hydroxy-3-methyl-N-((1 S)-1-methyl-2-{[(1S)-3-methyl-2-oxo-2,3,4,5-tetrahydro-1H-3-benzazepin-1-yl]amino}-2-oxoethyl)butanamide); Examples of gamma secretase inhibitors include, but are not limited to, DBZ (Axon Medchem, Cat. No. 1488), BMS-906024 (Bristol-Myers Squibb), R04929097 (Roche/Genentech), LY450139 (Eli Lilly), BMS-708163 (Bristol-Myers Squibb), MK-0752 (University of Michigan), PF-03084014 (Pfizer), IL-X (also referred to as cbz-IL-CHO, Calbiochem), z-Leu-leu-Nle-CHO (EMD Millipore), N-[N-(3,5-difluorophenacetyl)-L-alanyl]-Sphenylglycine t-butyl ester (DAPT), BH589 (Panobinostat, Novartis), MEDI0639 (MedImmune LLC), Choline magnesium trisalicylate (e.g., Trilisate), and Curcumin (a curcuminoid of turmeric). In one embodiment, a Notch inhibitor provided as a part of a plurality of small molecules can be DAPT, also known as N—[N-(3,5-Difluorophenacetyl)-L-alanyl]-S-phenylglycine t-butyl ester. Derivatives and/or pharmaceutically acceptable salts of the Notch inhibitor may also be provided.

In addition, Notch inhibitors include antisense nucleic acids; RNA interfering molecules (e.g., siRNA); dominant-negative variants against a Notch transcript; and expression vectors thereof. Examples of these nucleotide based inhibitors are commercially available such as from ThermoFisher Scientific, inter alia, and described in PCT Pub. WO2005/042705 and U.S. Pat. Pub 2012/0322857, US Pat. Pub 2007/0093440; and Okuhashi et al. Anticancer Research October 2013 vol. 33 no. 10 4293-4298.

### Rock Inhibitors

A ROCK inhibitor is not particularly limited, provided that it can inhibit functions of Rho kinase (ROCK). Examples thereof include: Y-27632 ((+)-(R)-trans-4-(1-aminoethyl)-N-(4-pyridyl)cyclohexanecarboxamide dihydrochloride) (e.g., Ishizaki et al., Mol. Pharmacol., 57, 976-983, 2000; Narumiya et al., Methods Enzymol., 325, 273-284, 2000); Fasudil/HA1077 (e.g., Uenata et al., Nature 389: 990-994, 1997); H-1152 (e.g., Sasaki et al., Pharmacol. Ther., 93: 225-232, 2002); Wf-536 (e.g., Nakajima et al., Cancer Chemother. Pharmacol., 52 (4): 319-324, 2003) and derivatives thereof; antisense nucleic acids against ROCK; RNA interfering molecules (e.g., siRNA); dominant-negative variants; and expression vectors thereof. Since other low-molecular-weight compounds are known as ROCK inhibitors, such compounds and derivatives thereof can also be used in the present invention (e.g., U.S. Patent Application Publication Nos. 2005/0209261, 2005/0192304, 2004/0014755, 2004/0002508, 2004/0002507, 2003/0125344, and 2003/0087919, WO 2003/062227, WO 2003/059913, WO 2003/062225, WO 2002/076976, and WO 2004/039796). In the present invention, one or more types of ROCK inhibitors can be used.

Within the context of the current disclosure a ROCK-inhibitor comprises both an inhibitor of ROCK1 and/or of ROCK2. Rho-associated protein kinase (ROCK) is a kinase belonging to the AGC (PKA/PKG/PKC) family of serine-threonine kinases. It is mainly involved in regulating the shape and movement of cells by acting on the cytoskeleton. Details on ROCKs, and their function are reviewed by Morgan-Fisher et al (2013) J Histochem Cytochem 61(3) 185-198. The two ROCKs, ROCK I (also known as p160ROCK and ROKβ) and ROCK II (Rho-kinase and ROKα), are 160-kDa proteins encoded by distinct genes. The mRNA of both kinases is ubiquitously expressed, but the ROCK I protein is mainly found in organs such as liver, kidney, and lung, whereas ROCK II protein is mainly found in muscle and brain. The amino acid sequences of the two ROCKs are highly homologous (^{˜}65%), and they exhibit the same overall domain structure.

The ROCKs were first identified almost 20 years ago and were suggested to be regulators of the actin cytoskeleton downstream of Rho. Since then, a range of interaction partners for ROCKs have been identified, many of which are linked to regulation of the actin cytoskeleton, including ezrin/radixin/moesin (ERM), the LIM-kinases (LIMK), myosin light chain (MLC), and MLC-phosphatase (MLCP).

By ROCK activity is meant any function of ROCK, such as regulation of the cytoskeleton through the phosphorylation of downstream substrates, leading to increased actin filament stabilization and generation of actin-myosin contractility.

Mammalian cells encode two Rho kinases, ROCK1 and ROCK2. These kinases are activated by binding to an active, GTP-bound Rho GTPase. Accordingly, reference to a ROCK protein herein comprises ROCK1 and ROCK2. As discussed above, ROCK phosphorylates a number of substrates on serine or threonine residues. These substrates are involved in a wide range of cell behavior. For example, myosin light chain phosphatase, involved in stress fiber formation and contractility; LIM kinase, involved in actin stabilization; NHE1 involved in focal adhesions and actin; and PTEN and Ezrin (Mueller et al., Nat. Rev. Drug Discov. 4:387-398, 2005; Riento et al., Nat. Rev. Mol. Cell Biol. 4:446-456, 2003). ROCK inhibitors such as Y-27632 and Fasudil bind to the catalytic site in the kinase domain and displace ATP.

ROCK inhibitors are known to those skilled in the art, and such inhibitors as suggested in the art are described herein and are in use in clinical trials for the treatment of several clinical conditions. These include Fasudil which is currently in use in Japan for treatment of cerebral vasospasm after subarachnoid hemorrhage. Other ROCK inhibitors have been through phase I and II trials for glaucoma and spinal cord injury, examples include Wf-536, Y-27632, and RKI-1447 and Slx-2119.

In one embodiment, the ROCK inhibitor is a small molecule. Exemplary small molecule ROCK inhibitors described in the art include Y-27632 (U.S. Pat. No. 4,997,834) and Fasudil (also known as HA 1077; Asano et al., J. Pharmacol. Exp. Ther. 241:1033-1040, 1987).

Other small molecules reported to specifically inhibit ROCK include H-1152 ((S)-(+)-2-Methyl-1-[(4-methyl-5-isoquinolinyl)sulfonyl]homopiperazine, Ikenoya et al., J. Neurochem. 81:9, 2002; Sasaki et al., Pharmacol. Ther. 93:225, 2002); N-(4-Pyridyl)-N′-(2,4,6-trichlorophenyl)urea (Takami et al., Bioorg. Med. Chem. 12:2115, 2004); and 3-(4-Pyridyl)-1H-indole (Yarrow et al., Chem. Biol. 12:385, 2005).

Additional small molecule Rho kinase inhibitors include those described in WO 03/059913, WO 03/064397, WO 05/003101, WO 04/112719, WO 03/062225, WO 07/042321 and WO 03/062227; U.S. Pat. Nos. 7,217,722 and 7,199,147; and U.S. 2003/0220357, U.S. 2006/0241127, U.S. 2005/0182040 and U.S. 2005/0197328; and EP2542528, EP2597953. A non-limitative overview of well-known ROCK inhibitors is provided in Table 1 (Some of which also described in: Fasudil: Ying et al., Mol. Cancer Ther. 5:2158, 2006; Y27632: Routhier et al., Oncol. Rep. 23:861, 2010; Y39983: Tanihara et al., Clin. Sciences 126: 309, 2008; RKI-1447: Patel et al., Cancer Res. 72: 5025, 2012; GSK269962A: Doe et al., J Pharm. Exp. Ther. 320: 89, 2007).

Further examples of ROCK inhibitors that may be implemented in accord with the teachings include, but are not limited to, AMA-0076; AMA-0247; AR-12286; AR-13324; AS-1892802; ATS-8535; ATS-907; BA-1037; BA-1049; CCG-1423 (CAS No. 285986-88-1); Cethrin; DE-104; GSK2699662 (CAS No. 850664-21-0); GSK429286 (CAS No. 864082-47-3); H1152P (CAS No. 451462-58-1); HA1077 (Fasudil; CAS No. 103745-39-7); HA1100 (CAS No. 105628-72-6); hydrochloride (hydroxyfasudil); HMN-1152; K-115; Ki-23095; Rho Inhibitor (C₂₀H₁₈N₆O); Rhosin; Rho kinase (Kalypsys/Alcon) inhibitor (IDDBCP260624); rho kinase inhibitor (Bayer); Rho Kinase Inhibitor II (CAS No. 97627-27-5); Rho Kinase Inhibitor III (CAS No. 7272-84-6); Rho Kinase Inhibitor IV (CAS No. 913844-45-8); Rho Kinase Inhibitor V (CAS No. 1072906-02-5); Rho Kinase Inhibitor VII (C₂₁H₂₄N₈); Rho kinase Inhibitors (Amakem/Halo; BioConsulting; Kowa); Rhostatin; RKI1447 (ROCKInhibitor XIII; CAS No. 1342278-01-6); ROCK inhibitor (Devgen); ROCK inhibitors (Bayer-Schering Pharma); ROKalpha inhibitors (BioFocus); SAR407899; SB772077B (CAS No. 607373-46-6); dihydrochloride SR 3677 (CAS No. 1072959-67-1); dihydrochloride Thiazovivin (CAS No. 1226056-71-8); WF-536 (CAS No. 539857-64-2); XD-4000 series; Y27632 (CAS No. 146986-50-7); Slx-2119; and/or Y39983 (CAS No. 471843-75-1).

Other examples of ROCK inhibitors include those described in the international patent publications WO98/06433, WO00/09162, WO00/78351, WO01/17562, WO02/076976, EP1256574, WO02/100833, WO03/082808, WO2004/009555, WO2004/024717, WO2004/108724, WO2005/003101, WO2005/035501, WO2005/035503, WO2005/035506, WO2005/058891, WO2005/074642, WO2005/074643, WO2005/080934, WO2005/082367, WO2005/082890, WO2005/097790, WO2005/100342, WO2005/103050, WO2005/105780, WO2005/108397, WO2006/044753, WO2006/051311, WO2006/057270, WO2006/058120, WO2006/072792WO2011107608A1, and WO2007026920A2.

In certain examples, the ROCK inhibitor is a small interfering nucleotide sequence capable of inhibiting ROCK activity, such as siRNA using one or more small double stranded RNA molecules. For example, ROCK activity in a cell can be decreased or knocked down by exposing (once or repeatedly) the cell to an effective amount of the appropriate small interfering nucleotide sequence. The skilled person knows how to design such small interfering nucleotide sequence, for example as described in handbooks such as Doran and Helliwell RNA interference: methods for plants and animals Volume 10 CABI 2009. A variety of techniques can be used to assess interference with ROCK activity of such small interfering nucleotide sequence, such as described in WO 2005/047542, for example by determining whether the candidate small interfering nucleotide sequence decreases ROCK activity. Candidate small interfering nucleotide sequences that are capable of interference may be selected to further analysis to determine whether they also inhibit proliferation of melanoma cells, for example by assessing whether changes associated with inhibition of proliferation of melanoma cells occurs in melanoma cells. Examples of nucleotide based inhibitors of ROCK are commercially available from ThermoFisher Scientific and Santa Cruz Biotech, for example. Other examples of known nucleotide based inhibitors are described in PCT Pub WO2006/053014; PCT Pub WO2010/065907, and EP2628482A1.

### Antisense and RNA Interfering Molecules

It has been noted that Foxo inhibitors, Notch inhibitors or ROCK inhibitors may include antisense nucleic acids (DNA or RNA); interfering RNAs such as small interfering RNA (siRNA) or shRNA, microRNAs or ribozymes to reduce or inhibit expression and hence the biological activity of the targeted proteins. Based on the known sequences of the targeted Foxo, Notch and ROCK proteins and genes encoding them, antisense DNA or RNA that are sufficiently complementary to the respective gene or mRNA to turn off or reduce expression can be readily designed and engineered, using methods known in the art. In a specific embodiment, antisense or siRNA molecules for use in the present invention are those that bind under stringent conditions to the targeted mRNA or targeted gene encoding one or more Foxo proteins identified by the Genbank numbers, or to variants or fragments that are substantially homologous to the mRNA or gene encoding one or more Foxo, Notch or ROCK proteins. Examples of antisense molecules, siRNA or shRNA that target Foxo proteins are provided in U.S. Pat. Nos. 8,580,948; and 9,457,079, inter alia, which are incorporated by reference.

Methods of making antisense nucleic acids are well known in the art. Further provided are methods of reducing the expression of one or more Foxo, Notch or ROCK genes and mRNA in non-insulin producing gut cells by contacting the cells in situ or contacting isolated enriched populations of the cells or tissue explants in culture that comprise the cells with one or more of the antisense compounds or compositions of the invention. As used herein, the terms “target nucleic acid” encompass DNA encoding a Foxo, Notch or ROCK protein and RNA (including pre-mRNA and mRNA) transcribed from such DNA. The specific hybridization of a nucleic acid oligomeric compound with its target nucleic acid interferes with the normal function of the target nucleic acid. This modulation of function of a target nucleic acid by compounds which specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, and catalytic activity which may be engaged in or facilitated by the RNA. The overall effect of such interference with target nucleic acid function is modulating or reducing the expression of the protein encoded by the DNA or RNA. In the context of the present invention, “modulation” means reducing or inhibiting in the expression of the gene or mRNA for one or more Foxo proteins.

The targeting process includes determination of a site or sites within the target DNA or RNA encoding the Foxo, Notch or ROCK protein for the antisense interaction to occur such that the desired inhibitory effect is achieved. Within the context of the present invention, a preferred intragenic site is the region encompassing the translation initiation or termination codon of the open reading frame (ORF) of the mRNA for the targeted proteins. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine in eukaryotes. It is also known in the art that eukaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene. Routine experimentation will determine the optimal sequence of the antisense or siRNA.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, nucleic acids are chosen which are sufficiently complementary to the target; meaning that the nucleic acids will hybridize sufficiently well and with sufficient specificity, to give the desired effect of inhibiting gene expression and transcription or mRNA translation.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides which can hydrogen bond with each other. Thus, :“specifically hybridizable” and “complementary” are terms which are used to indicate a sufficient degree of complementarity or precise pairing such that stable and specific binding occurs between the nucleic acid and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of function, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

The antisense compounds in accordance with the teachings herein may comprise from about 8 to about 50 nucleobases (i.e., from about 8 to about 50 linked nucleosides). In specific embodiments, the antisense compounds are antisense nucleic acids comprising from about 12 to about 30 nucleobases. Alternatively, antisense compounds pertain to ribozymes, external guide sequence (EGS) nucleic acids (oligozymes), and other short catalytic RNAs or catalytic nucleic acids which hybridize to the target nucleic acid and modulate its expression. Nucleic acids in the context of this invention include “oligonucleotides,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Antisense nucleic acids have been employed as therapeutic moieties in the treatment of disease states in animals and man Antisense nucleic acid drugs, including ribozymes, have been safely and effectively administered to humans and numerous clinical trials are presently underway. It is thus established that nucleic acids can be useful therapeutic modalities that can be configured to be useful in treatment regimes for treatment of cells, tissues and animals, especially humans, for example to down-regulate expression of a Foxo, Notch or ROCK proteins.

The antisense and siRNA compounds can be utilized for diagnostics, therapeutics, and prophylaxis and as research reagents and kits. For therapeutics, an animal, preferably a human, suspected of having a disease or disorder such as diabetes, metabolic syndrome, glucose intolerance, and/or obesity where there is an inappropriately low level of insulin, which can be treated by reducing the expression of a Foxo, Notch or ROCK protein, is treated by administering antisense compounds in accordance with the teachings herein. The compounds can be utilized in pharmaceutical compositions by adding an effective amount of an antisense compound to a suitable pharmaceutically acceptable diluent or carrier. The antisense compounds and methods of the invention are useful prophylactically, e.g., to prevent or delay the appearance of diabetes, glucose intolerance, metabolic syndrome or obesity. The antisense compounds and methods of the invention are also useful to retard the progression of metabolic syndrome, glucose intolerance, diabetes, atherosclerosis or obesity.

While antisense nucleic acids are the typical form of antisense compound, the present disclosure comprehends other oligomeric antisense compounds, including but not limited to oligonucleotide mimetics.

The present invention also includes pharmaceutical compositions and formulations which include the antisense compounds described herein. The term “formulation” is encompassed by the term composition.

In mammalian cell culture, a siRNA-mediated reduction in gene expression has been accomplished by transfecting cells with synthetic RNA nucleic acids (Caplan et al., 2001; Elbashir et al., 2001). The 2004/0023390 application, the entire contents of which are hereby incorporated by reference as if fully set forth herein, provides exemplary methods using a viral vector containing an expression cassette containing a pol II promoter operably-linked to a nucleic acid sequence encoding a small interfering RNA molecule (siRNA) targeted against a gene of interest.

Certain embodiments are directed to the use of shRNA, antisense or siRNA to block expression of FOXO1, 3 and/or 4, Notch or ROCK or orthologs, analogs and variants thereof in an animal. Antisense nucleotides can be designed using routine skill in the art to target human DNA or mRNA encoding a FOXO, Notch or ROCK protein as is described in more detail below. The antisense compounds of the invention are synthesized in vitro and do not include antisense compositions of biological origin, or genetic vector constructs designed to direct the in vivo synthesis of antisense molecules.

There are various embodiments to deliver antisense or RNA interfering molecules to gut cells. There are tested delivery methods to achieve in vivo transfection such as coating siRNA with liposomes or nanoparticles. There is also a novel technology that specifically targets siRNA delivery to gut epithelium, called “Transkingdom RNA interference.” The inventors of this technique have genetically engineered non-pathogenic E. coli bacteria that are able to produce short hairpin RNA (shRNA) targeting a mammalian gene (Xiang, S., et al., 2009. In vitro and in vivo gene silencing by TransKingdom RNAi (tkRNAi). Methods Mol Biol 487:147-160.). Two factors were used to facilitate shRNA transfer: the invasin (Inv) and listeriolysin O (HlyA) genes. They have shown that the recombinant E. coli can be administered orally to deliver an shRNA against Catenin b1 (Ctnnb1) that inhibits expression of this gene in intestinal epithelial cells without demonstrable systemic complications from leaking of bacteria into the bloodstream. Certain embodiments of the invention are directed to using the Transkingdom RNA interference method adapted to siRNA that silences one or more Foxo proteins.

Others have used this technique to knock down Abcb1 (Kruhn, A., et al., 2009. Delivery of short hairpin RNAs by transkingdom RNA interference modulates the classical ABCB1-mediated multidrug-resistant phenotype of cancer cells. Cell Cycle 8).

In one specific example, bacteria encoding the Foxo1 shRNA can be purchased from Cequent Technologies, and can be administered inter alia it by oral gavage at the recommended concentrations. Doses can be determined using analysis of Foxo1 knock-down in intestinal cells in biopsies, for example or in test animals.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Chimeric antisense compounds may be formed as composite structures of two or more oligonucleotides, modified oligonucleotides, oligonucleotides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

The antisense nucleic acid or RNA interfering molecules are typically administered to a subject or generated in situ such that they hybridize sufficiently with or bind to cellular mRNA and/or genomic DNA encoding the protein of interest to thereby reduce expression of the protein, e.g., by reducing transcription and/or translation. The hybridization can be by conventional nucleotide complementary to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules or RNA interfering molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens.

The antisense nucleic acid molecules or RNA interfering molecules can also be delivered to cells using the vectors described herein. To achieve sufficient intracellular concentrations of the antisense molecules, vector constructs in which the antisense nucleic acid molecule or interfering RNA molecule may be placed under the control of a strong pol II or pol III promoter.

An antisense nucleic acid molecule for use herein can be an alpha-anomeric nucleic acid molecule. An α-anomeric nucleic acid molecule forms specific double-stranded hybrids with complementary RNA in which, contrary to the usual βunits, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330). All of the methods described in the above articles regarding antisense technology are incorporated herein by reference.

Inhibitor embodiments also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave targeted mRNA transcripts thereby inhibiting translation. A ribozyme having specificity for a targeted-encoding nucleic acid can be designed based upon the nucleotide sequence of its cDNA. For example, a derivative of a Tetrahymena L-19 IVS RNA can be constructed in which the nucleotide sequence of the active site is complementary to the nucleotide sequence to be cleaved in the targeted mRNA. See, e.g., Cech et al. U.S. Pat. No. 4,987,071; and Cech et al. U.S. Pat. No. 5,116,742. Alternatively, a targeted FOXO, Notch or ROCK mRNA can be used to select a catalytic RNA having a specific ribonuclease activity from a pool of RNA molecules. See, e.g., Bartel and Szostak (1993) Science 261:1411-1418, incorporated herein by reference.

As used herein, the term “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand). As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that flank an ARPKD gene). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered. Unless otherwise indicated, all nucleic acid sequences herein are given in the 5′ to 3′ direction. Also, all deoxyribonucleotides in a nucleic acid sequence are represented by capital letters (e.g., deoxythymidine is “T”), and ribonucleotides in a nucleic acid sequence are represented by lower case letters (e.g., uridine is “u”).

### Antibodies

Agents that reduce the biological activity of a Foxo protein, protein of the Notch pathway or ROCK include antibodies (including portions or fragments or variants of antibody fragments or variants of antibodies) that have specific binding affinity for the intended target, thereby interfering with its biological activity. These antibodies recognize an epitope in a target protein or biologically active fragment thereof, such as Foxo 1, 3 or 4, Notch or ROCK. In certain embodiments the antibodies reduce the ability of Foxo to increase N3 synthesis.

An “antibody” refers to an intact immunoglobulin or to an antigen-binding portion (fragment) thereof that competes with the intact antibody for specific binding, and is meant to include bioactive antibody fragments. Therapeutically useful antibodies in treating or preventing an enumerated disease or changing a phenotype as described include any antibody to any Foxo, Notch or ROCK protein or analog, ortholog or variant thereof, that reduces the biological activity of the respective target in a Gut Ins− cell, such as a Gut N3 Frog cell.

Once produced, antibodies or fragments thereof can be tested for recognition of the target polypeptide by standard immunoassay methods including, for example, enzyme-linked immunosorbent assay (ELISA) or radioimmunoassay assay (RIA). See, Short Protocols in Molecular Biology eds. Ausubel et al., Green Publishing Associates and John Wiley & Sons (1992).

The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids. The terms “antibody” and “antibodies” include polyclonal antibodies, monoclonal antibodies, humanized or chimeric antibodies, single chain Fv antibody fragments, Fab fragments, and F(ab′)₂ fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies are particularly useful in the present invention.

Antibody fragments that have specific binding affinity for the polypeptide of interest can be generated by known techniques. Such antibody fragments include, but are not limited to, F(ab′)₂ fragments that can be produced by pepsin digestion of an antibody molecule, and Fab fragments that can be generated by reducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

An “isolated antibody” is an antibody that (1) is not associated with naturally-associated components, including other naturally-associated antibodies, that accompany it in its native state, (2) is free of other proteins from the same species, (21) is expressed by a cell from a different species, or (4) does not occur in nature.

The term “human antibody” includes all antibodies that have one or more variable and constant regions derived from human immunoglobulin sequences. In a preferred embodiment, all of the variable and constant domains are derived from human immunoglobulin sequences (a fully human antibody). These antibodies may be prepared in a variety of ways, as described below.

A humanized antibody is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293, incorporated herein by reference.

The term “chimeric antibody” refers to an antibody that contains one or more regions from one antibody and one or more regions from one or more other antibodies.

Fragments, portions or analogs of antibodies can be readily prepared by those of ordinary skill in the art following the teachings of this specification. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991).

### Biologically Active Fragments or Variants of an Agent

Biologically active fragments or variants of the therapeutic agents are also within the scope of the present invention. As described herein, “biologically active” means, alone or in co-administration with other agents described herein, increasing at least one effect selected from the group comprising inducing mammalian Gut Ins− Cells to express insulin, increasing insulin sensitivity, increasing glucose tolerance, decreasing weight gain, decreasing fat mass, increasing weight loss in animals with impaired pancreatic function i.e. that do not make or secrete normal levels of insulin. Fragments and variants are described below. Fragments can be discrete (not fused to other amino acids or peptides) or can be within a larger peptide. Further, several fragments can be comprised within a single larger peptide.

Other variants of peptides include those that provide useful and novel characteristics for the agent. For example, the variant of a peptide agent may have reduced immunogenicity, increased serum half-life, increased bioavailability and/or increased potency. “Variants of peptide agents” refers to peptides that contain modifications in their amino acid sequences such as one or more amino acid substitutions, additions, deletions and/or insertions but that are still biologically active. In some instances, the antigenic and/or immunogenic properties of the variants are not substantially altered, relative to the corresponding peptide from which the variant was derived. Such modifications may be readily introduced using standard mutagenesis techniques, such as oligonucleotide directed site-specific mutagenesis as taught, for example, by Adelman et al. (DNA, 2:183, 1983) or by chemical synthesis. Variants and fragments are not mutually exclusive terms. Fragments also include peptides that may contain one or more amino acid substitutions, additions, deletions and/or insertions such that the fragments are still biologically active. Fully functional variants typically contain only conservative variation or variation in non-critical residues or in non-critical regions. Functional variants can also contain substitutions of similar amino acids, which results in no change, or an insignificant change, in function. Alternatively, such substitutions may positively or negatively affect function to some degree. The activity of such functional agent variants can be determined using assays such as those described herein.

Some variants are also derivatives of the agents. Derivatization is a technique used in chemistry which transforms a chemical compound into a product of similar chemical structure, called derivative. Generally, a specific functional group of the compound participates in the derivatization reaction and transforms the educt to a derivate of deviating reactivity, solubility, boiling point, melting point, aggregate state, functional activity, or chemical composition. Resulting new chemical properties can be used for quantification or separation of the educt or can be used to optimize the compound as a therapeutic agent. The well-known techniques for derivatization can be applied to the agents. Thus, derivatives of peptide agents described above will contain amino acids that have been chemically modified in some way so that they differ from the natural amino acids.

Provided also are agent mimetics. “Mimetic” refers to a synthetic chemical compound that has substantially the same structural and functional characteristics of a naturally or non-naturally occurring peptide, and includes, for instance, peptide- and polynucleotide-like polymers having modified backbones, side chains, and/or bases. Peptide mimetics are commonly used in the pharmaceutical industry as non-peptide drugs with properties analogous to those of the template peptide. Generally, mimetics are structurally similar (i.e., have the same shape) to a paradigm peptide that has a biological or pharmacological activity, but one or more peptide linkages are replaced. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

A brief description of various protein modifications that can be made to active agents that come within the scope of this invention are described in Karsenty, US Application 20100190697.

### Pharmaceutical Preparations

Certain embodiments of the present invention are directed to pharmaceutical compositions and formulations that include one or more enumerated agents as defined herein, including but not limited to small molecules, polypeptides, antibodies, nucleic acids (including antisense RNA, siRNA, microRNAs, Cop1 (Caspase recruitment domain-containing protein 16) and ribozymes that reduce the expression and/or biological activity of a FOXO, Notch or ROCK protein in Gut Ins− cells, thereby causing them to differentiate or convert into Gut Ins' Cells that make and secrete insulin. The term formulation refers to a composition that has two or more components and is typically formulated for a certain type of administration. The pharmaceutical compositions will have one or more of the following effects of increasing insulin secretion and serum insulin, increasing insulin sensitivity, increasing glucose tolerance, decreasing weight gain, decreasing fat mass, and causing weight loss.

The therapeutic agents are generally administered in an amount sufficient to treat or prevent diabetes type 1 and 2, metabolic syndrome, and obesity in a subject; or to reduce fat mass. The pharmaceutical compositions of the invention provide an amount of the active agent effective to treat or prevent an enumerated disease or disorder.

The candidate agent may be chemically modified to facilitate its uptake by Gut Ins− Cells. For example, it could be fused to a bile acid or fatty acid to facilitate uptake by gut cells; or it may be packaged in liposomes or another lipid-based emulsion system to facilitate its uptake; it may be encoded by bacteria expressing a modified cell surface antigen that promotes its binding to gut epithelial cells, including N3 Prog.cell-permeable peptides was used to improve cellular uptake. (Gratton et al., Nature Medicine 9, 357-362 (2003)).

The pharmaceutical compositions of the present invention may be administered in a number of ways depending upon whether local or systemic treatment is desired and upon the area to be treated. For example, certain gut regions known to have the highest density of Gut Ins− cells that can generate into Gut Ins+ cells can be targeted. Certain regions include, but are not limited to the ileum, duodenum, colon and rectum. Therefore, in some embodiments the pharmaceutical compositions are administered in formulations that target their release at the gut target region. Techniques for targeted delivery in the gut are well-known in the art. See for example Wikberg et al. Aliment Pharmacol Ther. 1997:11 (Suppl3):109-115; Dar et al., (2017) Polymer-based drug delivery: the quest for local targeting of inflamed intestinal mucosa, Journal of Drug Targeting, 25:7, 582-596; US Pat. Pubs 20050058701 and US20040224019; WO2014/152338; U.S. Pat. Nos. 7,670,627; 8,414,559; 9,023,368; and 9,730,884 all of which are incorporated by reference. Administration can also be intravenous, parenteral/intra-arterial, subcutaneous, intraperitoneal or intramuscular injection or infusion; or intracranial, e.g., intrathecal or intraventricular, administration. Suppositories can also be used. In some embodiments a slow release preparation comprising the active agents is formulated. The term “slow release” refers to the release of a drug from a polymeric drug delivery system over a period of time that is more than one day wherein the active agent is formulated in a polymeric drug delivery system that releases effective concentrations of the drug.

Certain medications, for example resins that prevent bile acid absorption, or inhibitors of sugar breakdown, are used in the treatment of type 2 diabetes and are not absorbed at all in the plasma. Such formulations are useful for the pharmaceutical formulations of the present invention.

The dosage administered, as single or multiple doses, to an individual will vary depending upon a variety of factors, including pharmacokinetic properties, subject conditions and characteristics (sex, age, weight, body mass index (BMI), general health), extent of symptoms, concurrent treatments, frequency of treatment and the effect desired. Not intended to be limiting, a dosage of the enumerated agent may range between 0.01 and 500 ng/mL, between 0.01 and 200 ng/mL, between 0.1 and 200 ng/mL, between 0.1 and 100 ng/mL, between 1 and 100 ng/mL, between 10 and 100 ng/mL, between 10 and 75 ng/mL, between 20 and 75 ng/mL, between 20 and 50 ng/mL, between 25 and 50 ng/mL, or between 30 and 40 ng/mL. In certain embodiments, the pharmaceutical compositions may comprise about 0.1 mg to 5 g, about 0.5 mg to about 1 g, about 1 mg to about 750 mg, about 5 mg to about 500 mg, or about 10 mg to about 100 mg of therapeutic agent.

In addition to continuous administration using osmotic pumps, active agents can be administered as a single treatment or, preferably, can include a series of treatments, that continue at a frequency and for a duration of time that causes one or more symptoms of the enumerated disease to be reduced or ameliorated, or that achieves the desired effect including effects of increasing insulin secretion and serum insulin, increasing insulin sensitivity, increasing glucose tolerance, decreasing weight gain, decreasing fat mass, and causing weight loss.

It is understood that the appropriate dose of an active agent depends upon a number of factors within the ken of the ordinarily skilled physician, veterinarian, or researcher. The dose(s) vary, for example, depending upon the identity, size, and condition of the subject or sample being treated, further depending upon the route by which the composition is to be administered, and the effect which the practitioner desires the an active agent to have. It is furthermore understood that appropriate doses of an active agent depend upon the potency with respect to the expression or activity to be modulated. Such appropriate doses may be determined using the assays described herein. When one or more of these active agents are to be administered to an animal (e.g., a human) in order to modulate expression or activity a Foxo protein, a relatively low dose may be prescribed at first, with the dose subsequently increased until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Type 1 diabetes is usually diagnosed in children and young adults—but can occur at any age, and was previously known as juvenile diabetes. In type 1 diabetes, the body does not produce insulin. Insulin is a hormone that is needed to convert sugar (glucose), starches and other food into energy needed for daily life. Conditions associated with type 1 diabetes include hyperglycemia, hypoglycemia, ketoacidosis and celiac disease.

Type 2 diabetes is the most common form of diabetes. In type 2 diabetes, either the body does not produce enough insulin or the cells ignore the insulin. Conditions associated with type 2 diabetes include hyperglycemia and hypoglycemia.

Disorders associated with energy metabolism include diabetes, glucose intolerance, decreased insulin sensitivity, decreased pancreatic beta-cell proliferation, decreased insulin secretion, weight gain, increased fat mass and decreased serum adiponectin.

The therapeutic agent can be formulated with an acceptable carrier using methods well known in the art. The actual amount of therapeutic agent will necessarily vary according to the particular formulation, route of administration, and dosage of the pharmaceutical composition, the specific nature of the condition to be treated, and possibly the individual subject. The dosage for the pharmaceutical compositions of the present invention can range broadly depending upon the desired effects, the therapeutic indication, and the route of administration, regime, and purity and activity of the composition.

A suitable subject can be an individual or animal that is suspected of having, has been diagnosed as having, or is at risk of developing an enumerated disease, and like conditions as can be determined by one knowledgeable in the art.

Techniques for formulation and administration can be found in “Remington: The Science and Practice of Pharmacy” (20.sup.th edition, Gennaro (ed.) and Gennaro, Lippincott, Williams & Wilkins, 2000), incorporated herein by reference. The pharmaceutical compositions of the present invention can be administered to the subject by a medical device, such as, but not limited to, catheters, balloons, implantable devices, biodegradable implants, prostheses, grafts, sutures, patches, shunts, or stents. A detailed description of pharmaceutical formulations of oligonucleotides is set forth in U.S. Pat. No. 7,563,884.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Enumerated agents may be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations disclosed herein, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of the ingredients enumerated above, as required, followed by filter sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. Depending on the specific conditions being treated, pharmaceutical compositions disclosed herein for treatment of atherosclerosis or the other elements of metabolic syndrome can be formulated and administered systemically or locally. Techniques for formulation and administration can be found in “Remington: The Science and Practice of Pharmacy” (20.sup.th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods as discussed above. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL®., or corn starch; a lubricant such as magnesium stearate or STEROTES®.; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams as generally known in the art.

If appropriate, the compounds can also be prepared in the form of suppositories (e.g., with conventional suppository bases such as cocoa butter and other glycerides) or retention enemas for rectal delivery.

In one embodiment, the enumerated agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art, for example, as described in U.S. Pat. No. 4,522,811.

It is especially advantageous to formulate oral or parenteral compositions in unit dosage form for ease of administration and uniformity of dosage. “Unit dosage form” as used herein refers to physically discrete units suited as unitary dosages for the subject to be treated; each unit containing a predetermined quantity of active agent calculated to produce the desired therapeutic effect in association with the required pharmaceutical carrier. The specification for the unit dosage forms are dictated by and directly dependent on the unique characteristics of the active agent and the particular therapeutic effect to be achieved, and the limitations inherent in the art of compounding such an active agent for the treatment of individuals.

As previously noted, the agent may be administered continuously by pump or frequently during the day for extended periods of time. In certain embodiments, the agent may be administered at a rate of from about 0.3-100 ng/hour, preferably about 1-75 ng/hour, more preferably about 5-50 ng/hour, and even more preferably about 10-30 ng/hour. The agent may be administered at a rate of from about 0.1-100 pg/hr, preferably about 1-75 micrograms/hr, more preferably about 5-50 micrograms/hr, and even more preferably about 10-30 micrograms/hr It will also be appreciated that the effective dosage of antibody, protein, or polypeptide used for treatment may increase or decrease over the course of a particular treatment. Changes in dosage may result and become apparent from monitoring the level of insulin and/or monitoring glycemia control in a biological sample, preferably blood or serum.

In an embodiment, the agent can be delivered by subcutaneous, long-term, automated drug delivery using an osmotic pump to infuse a desired dose of the agent for a desired time. Insulin pumps are widely available and are used by diabetics to automatically deliver insulin over extended periods of time. Such insulin pumps can be adapted to deliver the agent. The delivery rate of the agent to control glucose intolerance, diabetes types 1 or 2 can be readily adjusted through a large range to accommodate changing insulin requirements of an individual (e.g., basal rates and bolus doses). New pumps permit a periodic dosing manner, i.e., liquid is delivered in periodic discrete doses of a small fixed volume rather than in a continuous flow manner The overall liquid delivery rate for the device is controlled and adjusted by controlling and adjusting the dosing period. The pump can be coupled with a continuous blood glucose monitoring device and remote unit, such as a system described in U.S. Pat. No. 6,560,471, entitled “Analyte Monitoring Device and Methods of Use.” In such an arrangement, the hand-held remote unit that controls the continuous blood glucose monitoring device could wirelessly communicate with and control both the blood glucose monitoring unit and the fluid delivery device delivering enumerated agents.

The compositions may be formulated into any of many possible dosage forms such as, but not limited to, tablets, capsules, liquid syrups, soft gels, suppositories, and enemas. The compositions may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

### EXAMPLES

### Example 1. Co-Administration of Foxo1 Inhibitor (Compound 9) and Notch Inhibitor (DBZ)

The experiment consisted of performing surgery on 8-week-old mice to implant an enterojejujnal catheter to deliver drugs locally to the intestinal mucosa. After a 1-week recovery period, mice were treated with a single i.p. injection of DBZ or vehicle control. Administration of Foxo1 inhibitor compound 9 (Langlet et al. 2017 Cell, see below)

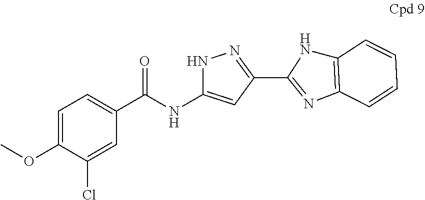 [SMILES: COc1ccc(C(=O)Nc2cc(-c3nc4ccccc4[nH]3)n[nH]2)cc1Cl]

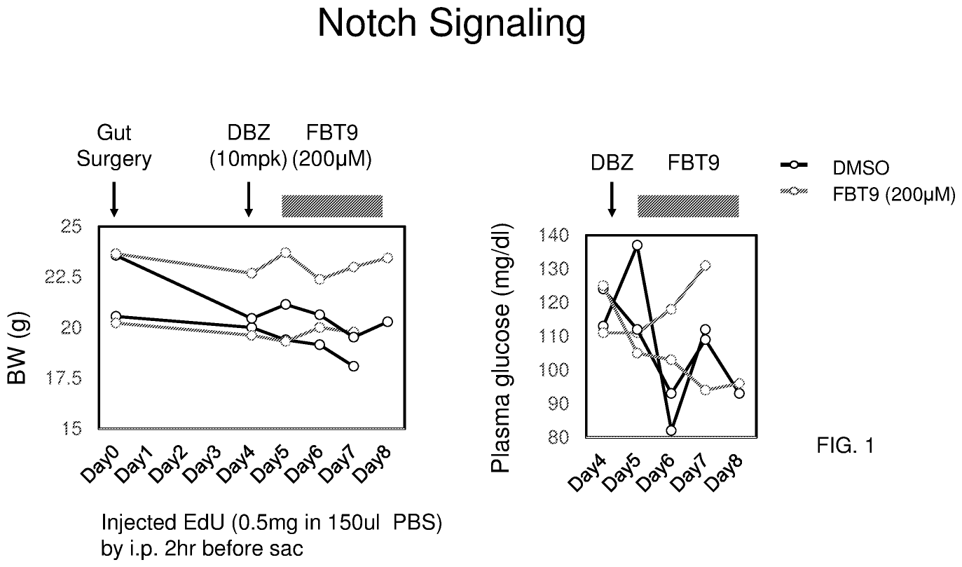

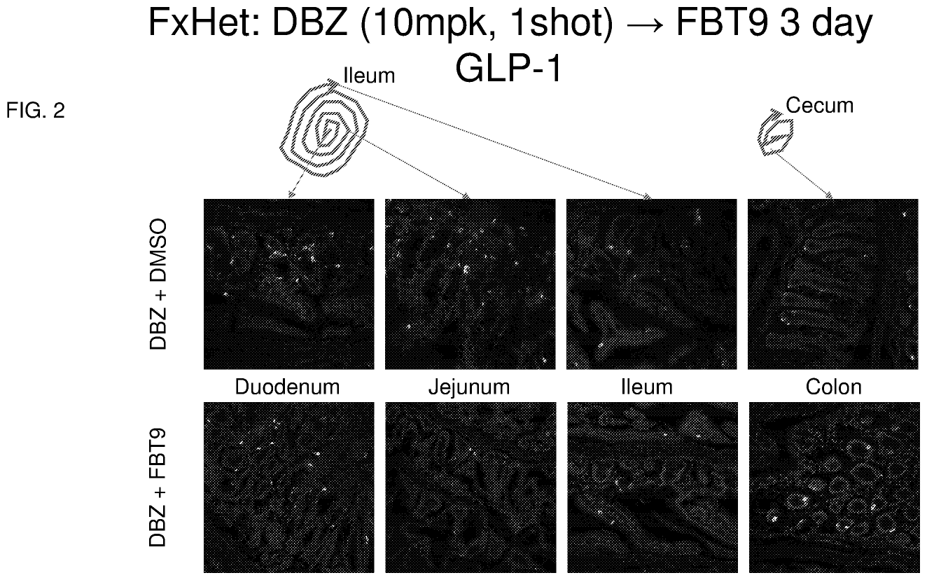

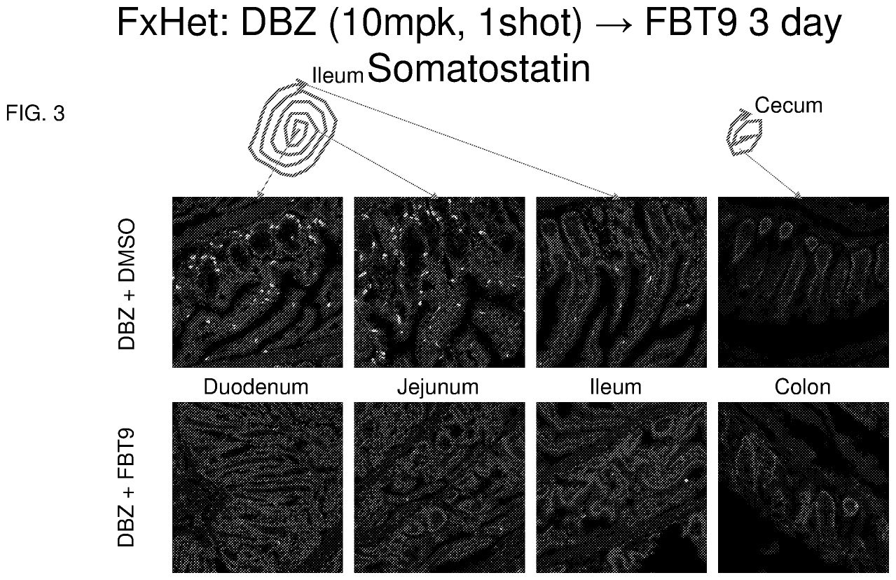

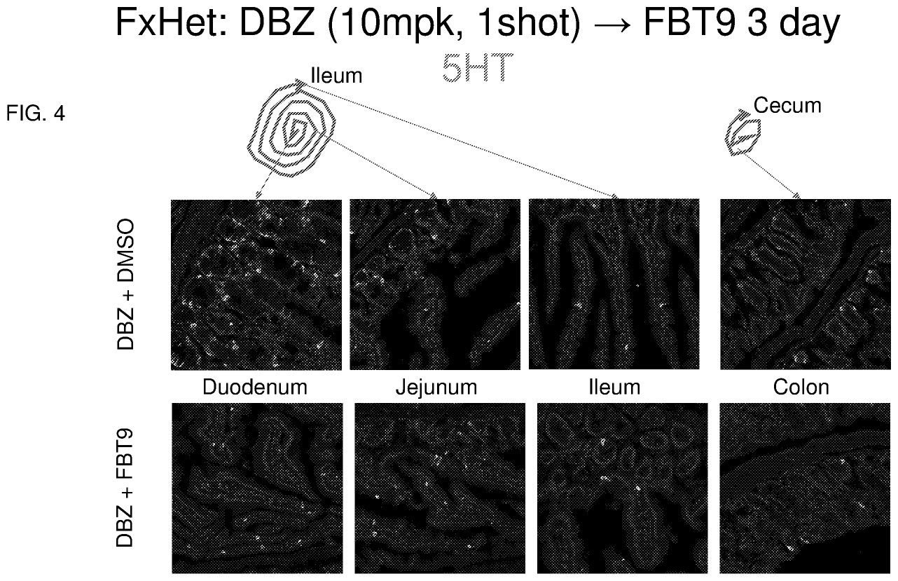

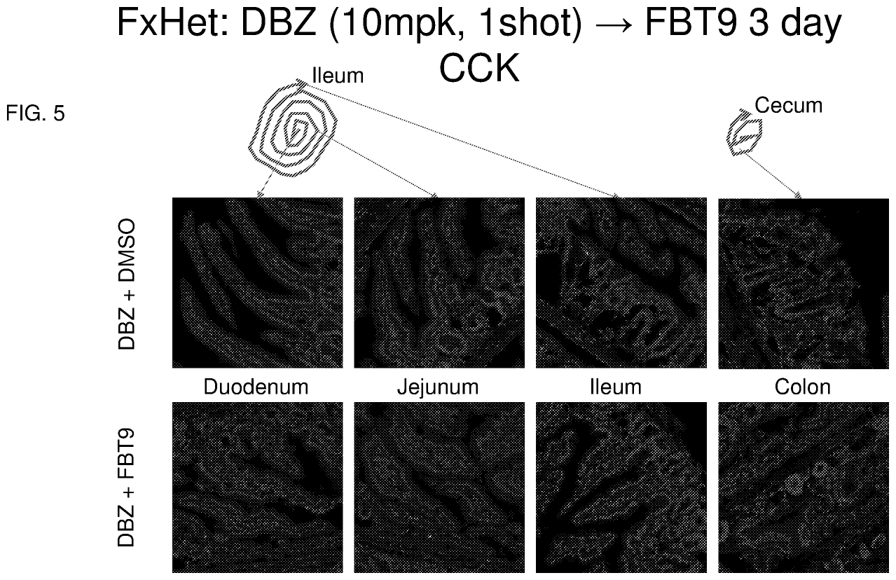

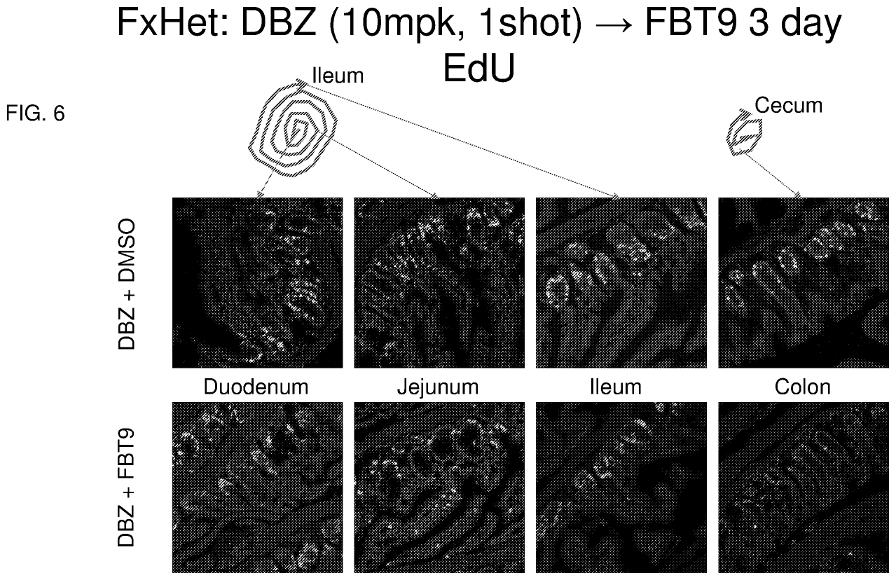

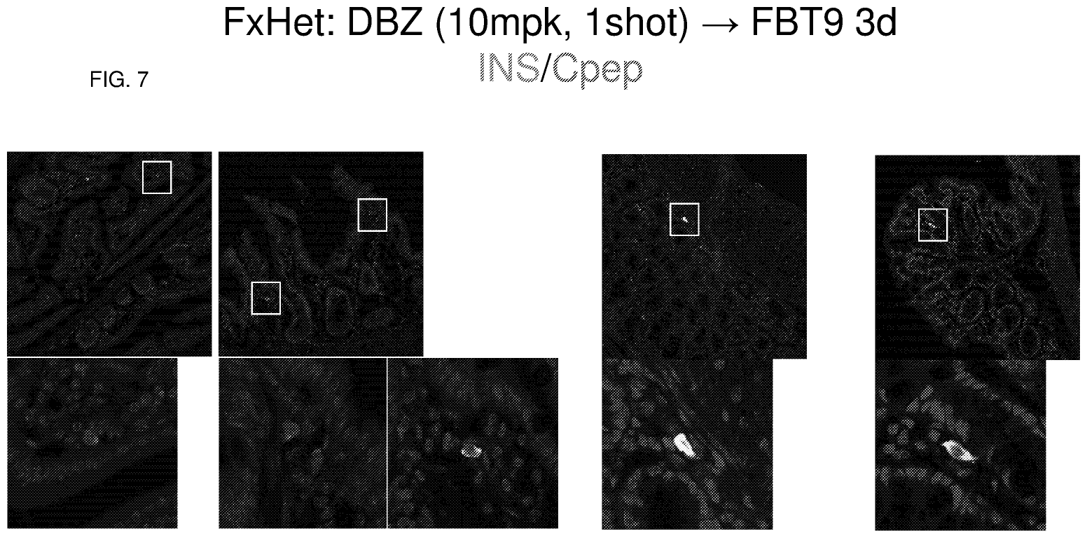

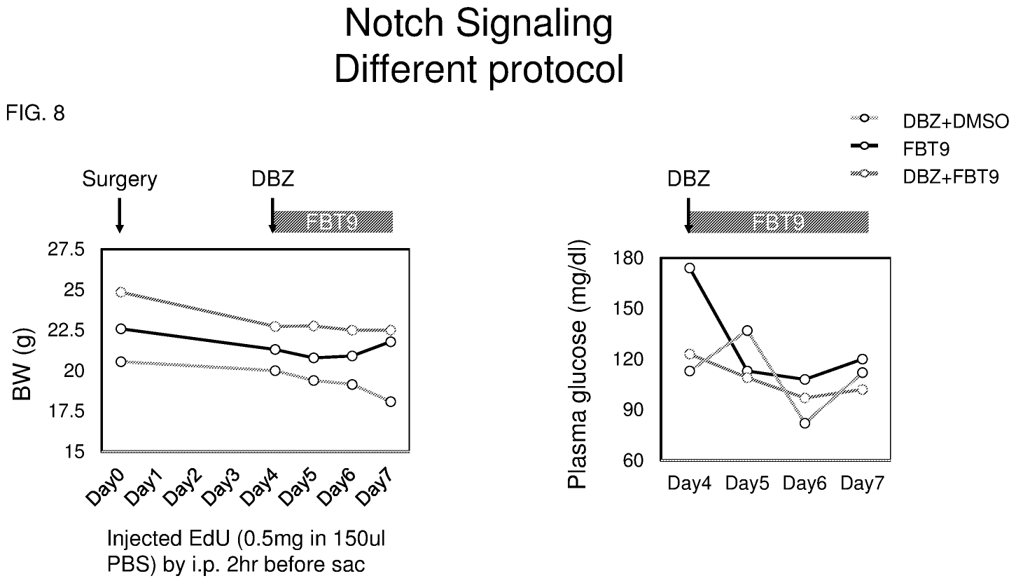

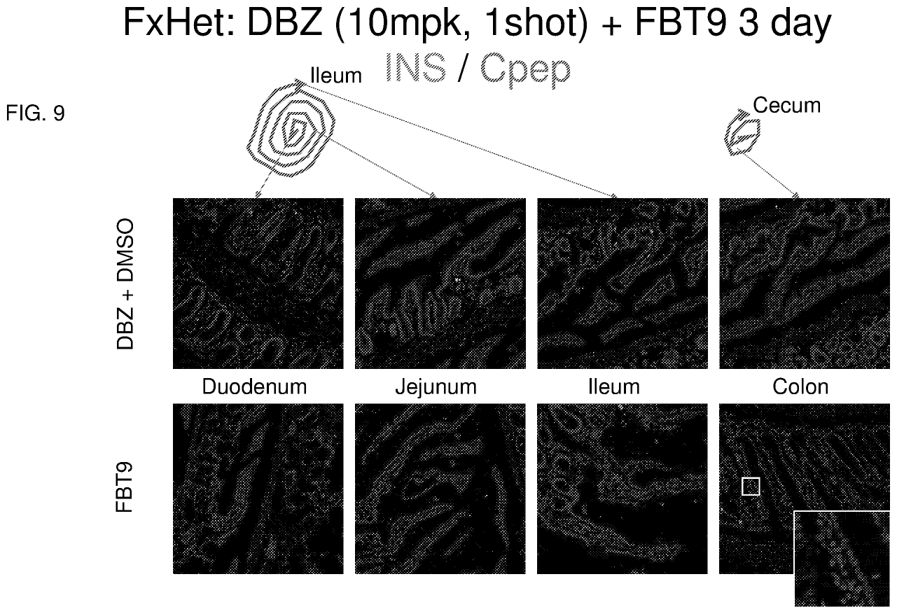

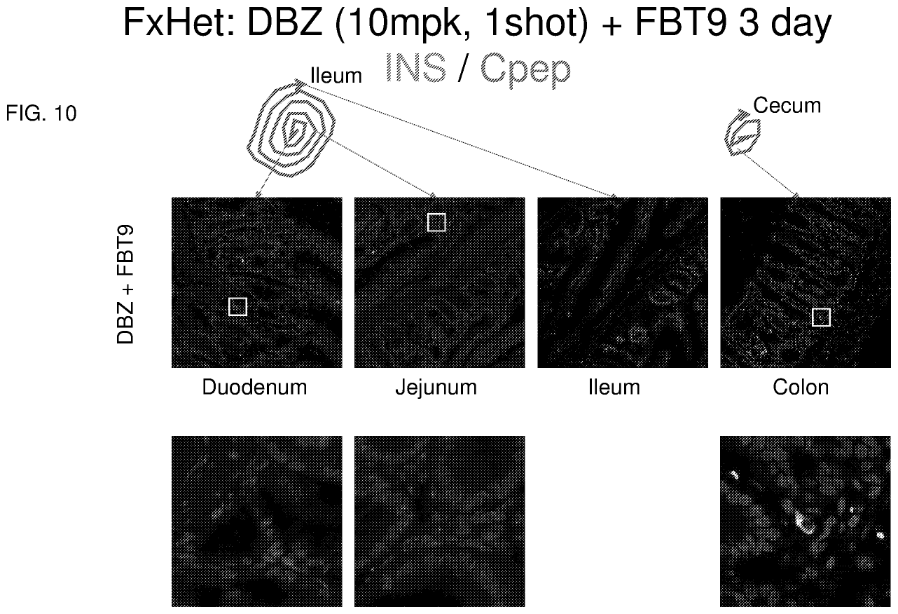

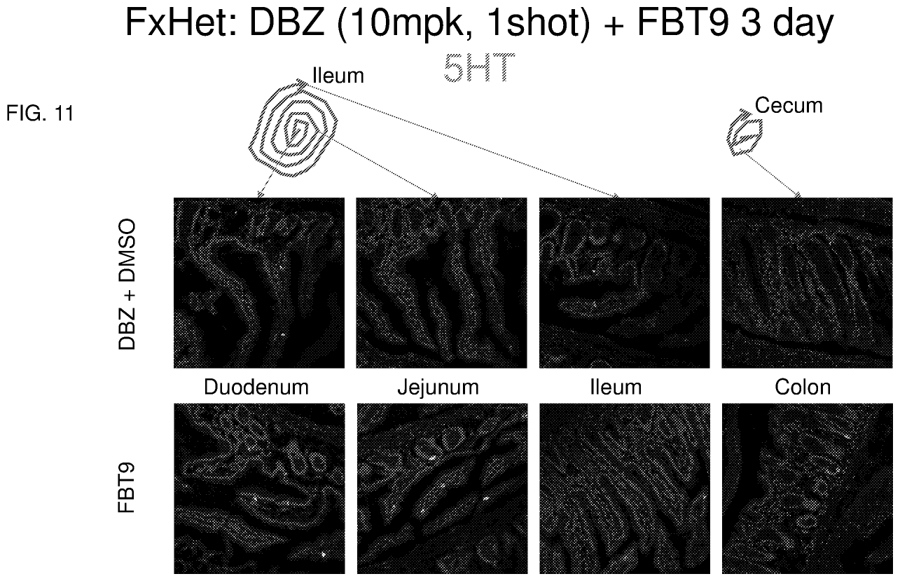

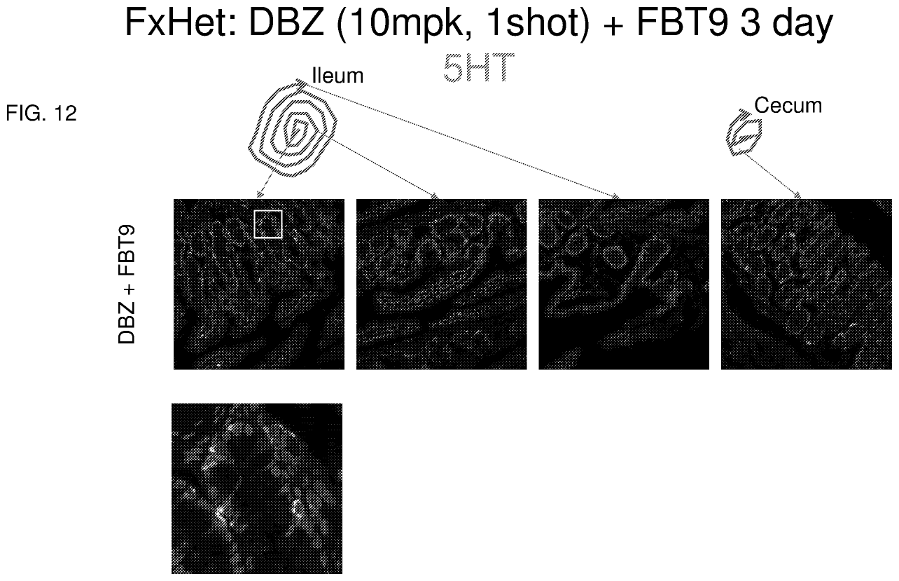

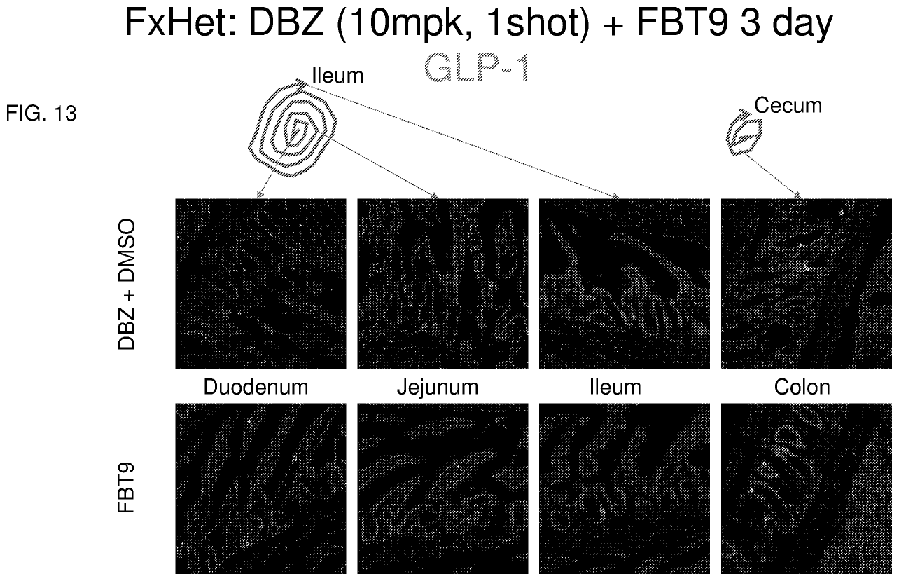

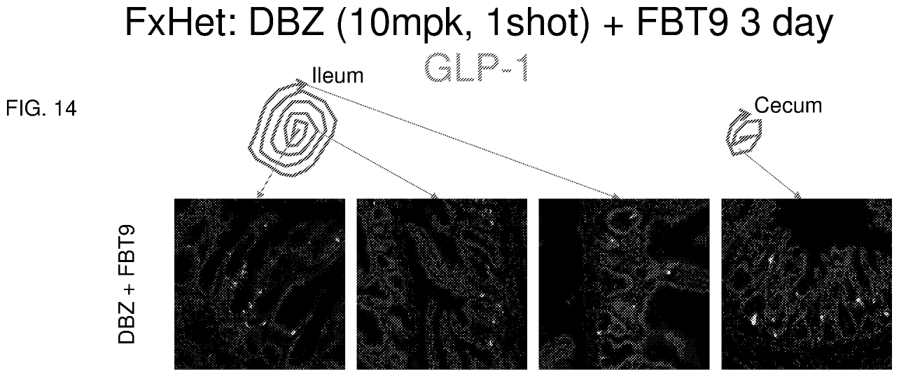

was initiated either on the same day or on the following day, t.i.d. by injection via enterojejunal catheter for 3 days. At the end of the experiment, mice were sacrificed and the intestine analyzed for enteroendocrine cell content using immunohistochemistry. The results of these experiments are provided in FIGS. 1-14. FIG. 7 shows that insulin-positive cells were generated by initial DBZ treatment and subsequent FBT9 treatment. FIG. 10 shows that the number of insulin-positive cells in the gut increased ˜5 fold over the treatment of FIG. 7. The treatment regime of FIG. 10 involved administering the first dose of FBT9 with DBZ followed by subsequent doses of FBT9.

### Example 2. Administration of ROCK Inhibitor in Foxo1 Knockout Mice

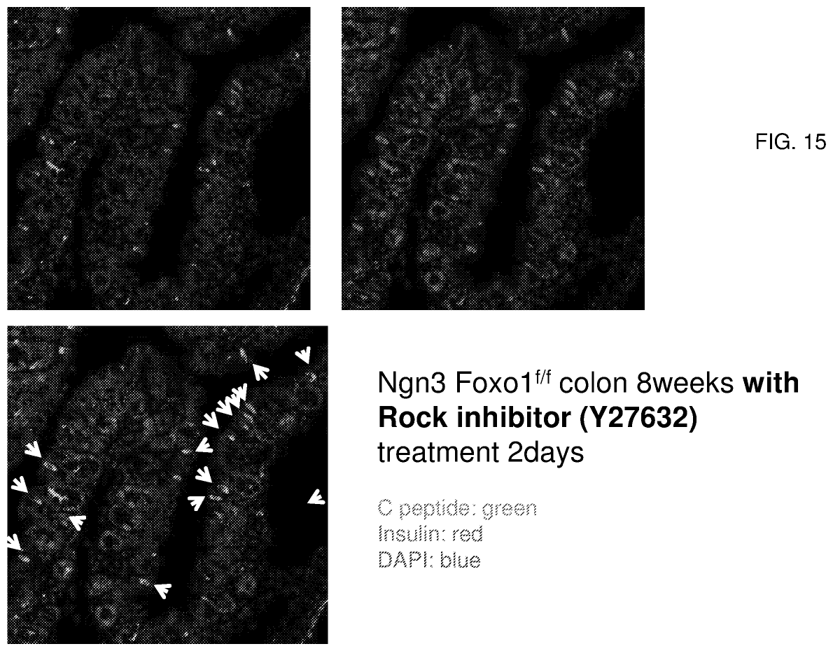

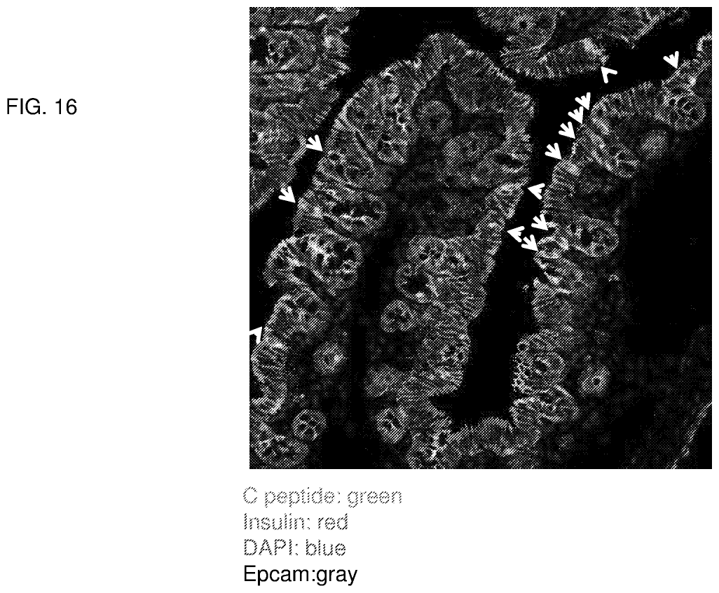

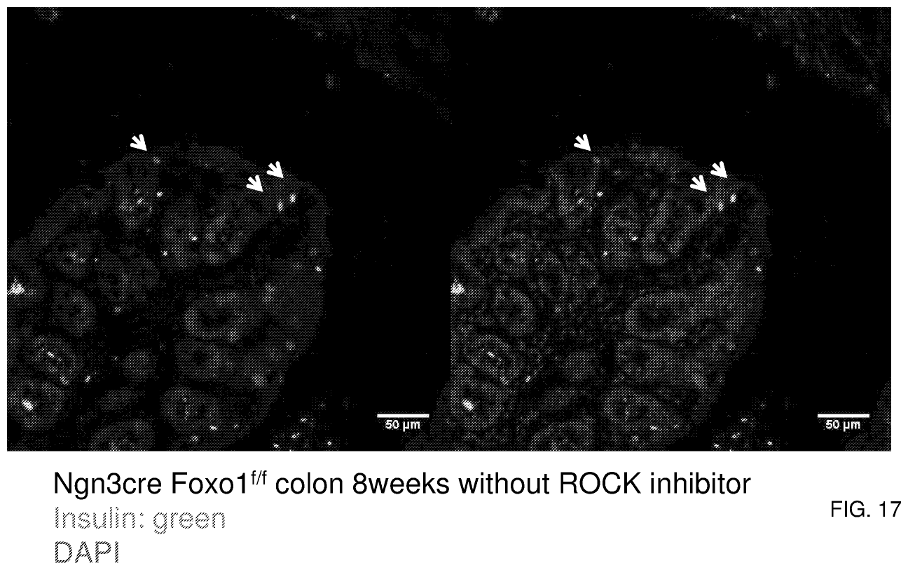

The experiment consisted of treating 8-week-old mice (Foxo1 knockout mice) by oral gavage dosing of Y-27632, q.d., for 2 days. On day 3, mice were sacrificed and the intestine analyzed for enteroendocrine cell content using immunohistochemistry. The results of these experiments are provided in FIGS. 15-17. The arrows in FIGS. 15 and 16 represent c-peptide and insulin-positive cells, which resemble true beta-like cells. FIG. 17 shows that the amount of insulin-positive cells decreases dramatically without treatment with ROCK inhibitor.

### Example 3. Administration of Foxo1 Inhibitor (Compound 10, “FBT10”) in Mouse Gut Organoid

Mouse gut organoid from a wild type mouse was treated with FBT10 (Compound 10, Langlet et al. 2017 Cell, see below).

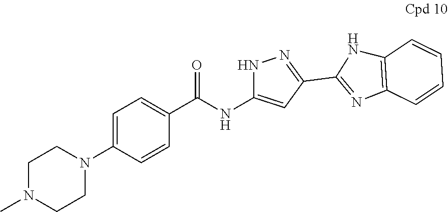 [SMILES: CN1CCN(c2ccc(C(=O)Nc3cc(-c4nc5ccccc5[nH]4)n[nH]3)cc2)CC1]

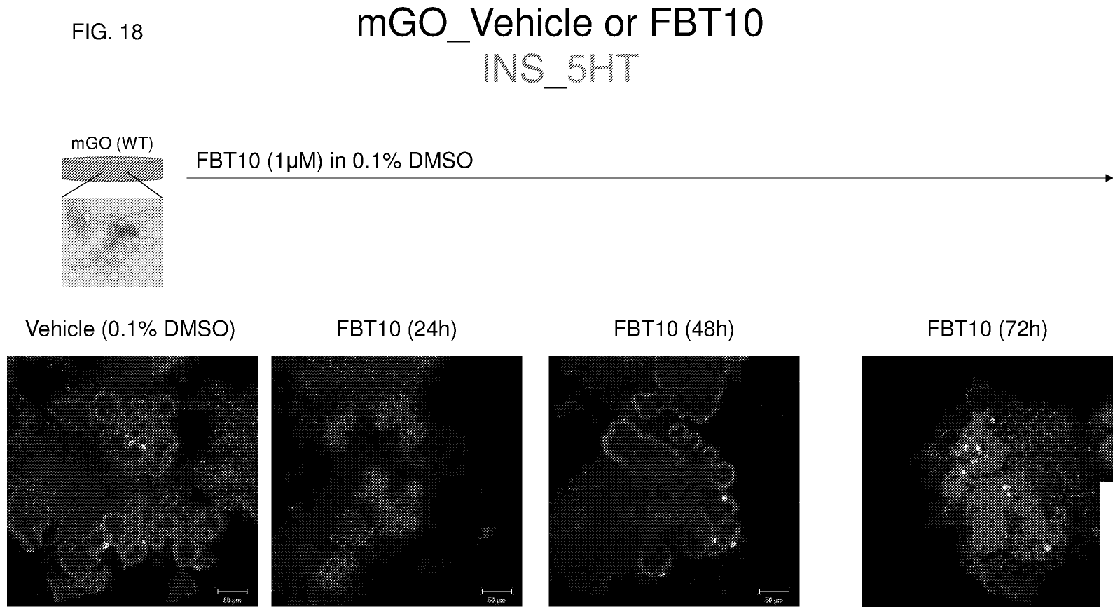

After 72 hrs of treatment, some of the cells turned into insulin and serotonin (5HT) positive cells confirmed by immunohistochemistry (see FIG. 18). This data demonstrates that FBT10 is capable of generating insulin-positive cells from gut cells.

### Example 4. Co-Administration of Foxo1 Inhibitor (FBT10) and Notch Inhibitor (DBZ)

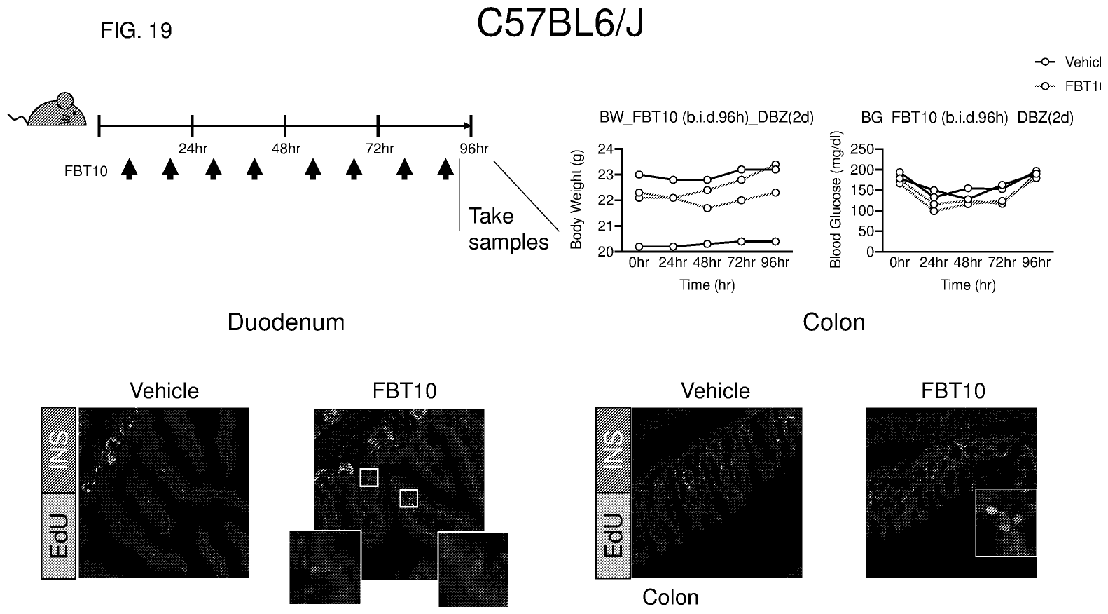

The protocol used above in Example 1 was followed for testing a combination of FBT10 and DBZ. The experiment consisted of performing surgery on 8-week-old mice to implant an enterojejujnal catheter to deliver drugs locally to the intestinal mucosa. After a 1-week recovery period, mice were treated with a single i.p. injection of DBZ or vehicle control. Administration of Foxo1 inhibitor compound 10 (FBT10) was initiated either on the same day or on the following day, t.i.d. by injection via enterojejunal catheter for 3 days. At the end of the experiment, mice were sacrificed and the intestine analyzed for enteroendocrine cell content using immunohistochemistry. The results of this experiment are provided in FIG. 19. Regarding the graphs indicating effects on Body weight and blood glucose, each line represents an individual animal. Insulin-positive cells were present in the duodenum and colon following FBT10 treatment. No insulin-positive cells were found in vehicle treated duodenum or colon.

### Example 5. Administration of FBT10 in NOD Mice

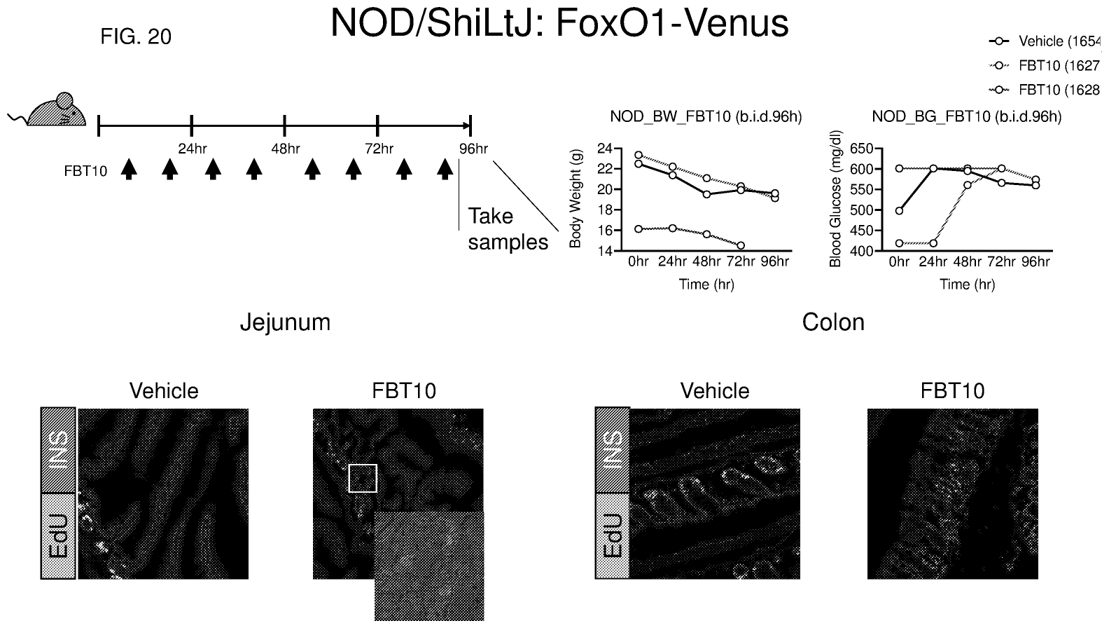

NOD mice (mouse model whose pancreatic beta cells are destroyed by immunological response) were treated with FBT10 or vehicle over a 96 hr period. The results of the experiment are shown in FIG. 20. As can be seen in the micrographs, FBT10 generated insulin-positive cells in the jejunum. Insulin-positive cells were not detected in the colon. FIG. 20 also provides graphs showing effects on body weight and blood glucose (each graph line represents an individual animal)

### Example 6

Examples of Foxo Antisense and RNA Interfering Molecules

short-hairpin RNA (from BD Biosciences)
GCACCGACTTTATGAGCAACC SEQ ID NO: 1
FOXO1-antisense
(TTG GGT CAG GCG GTT CA SEQ ID NO: 2);
FOXO3a-sense
(CCC AGC CTA ACC AGG GAA GT SEQ ID NO: 3)
FOXO3a-antisense
(AGC GCC CTG GGT TTG G SEQ ID NO: 4);
FOXO4-sense
(CCT GCA CAG CAA GTT CAT CAA SEQ ID NO: 5)
and
FOXO4-antisense
(TTC AGC ATC CAC CAA GAG CTT SEQ ID NO: 6)
Accell SMARTpool siRNA A-041127-13,
Target Sequence:
CUAUUAUUGUACAUGAUUG FOXO1 SEQ ID NO. 7
Mol. Wt. 13,501.1 (g/mol)
xt. Coeff. 372,198 (L/mol · cm)
Accell SMARTpool siRNA A-041127-14, FOXO1
Target Sequence:
CGAUGAUACCUGAUAAUG SEQ ID NO. 8
Mol. Wt. 13,521.4 (g/mol)
Ext. Coeff. 365,968 (L/mol · cm)
Accell SMARTpool siRNA A-041127-15, FOXO1
Target Sequence:
UCGUAAACCAUUGUAAUUA SEQ ID NO. 9
Mol. Wt. 13,489.3 (g/mol)
Ext. Coeff. 376,470 (L/mol · cm)
Accell SMARTpool siRNA A-041127-16, FOXO1
Target Sequence:
CCAGGAUAAUUGGUUUUAC SEQ ID NO. 10
Mol. Wt. 13,519.3 (g/mol)
Ext. Coeff. 361,874 (L/mol · cm)
R1-02, 5 nmol each of four controls +
delivery media
Catalog Item
K-005000-R1-02
Accell Mouse Control siRNA Kit-Red
The
ON-TARGETplus SMARTpool siRNA J-041127-05, FOXO1
Target Sequence:
GGUGUCAGGCUAAGAGUUA SEQ ID NO. 11
Mol. Wt. 13,429.9 (g/mol)
Ext. Coeff. 371,219 (L/mol · cm)
ON-TARGETplus SMARTpool siRNA J-041127-06, FOXO1
Mol. Wt. 13,414.8 (g/mol)
Ext. Coeff. 377,004 (L/mol · cm)
Target Sequence:
GUAAUGAUGGGCCCUAAUU SEQ ID NO. 12
ON-TARGETplus SMARTpool siRNA J-041127-07, FOXO1
Mol. Wt. 13,459.8 (g/mol)
Ext. Coeff. 357,691 (L/mol · cm)
Target Sequence:
GCAAACGGCUUCGGUCAAC SEQ ID NO. 13
ON-TARGETplus SMARTpool siRNA J-041127-08, FOXO1
Mol. Wt. 13,384.9 (g/mol)
Ext. Coeff. 384,302 (L/mol · cm)
Target Sequence:
GGACAACAACAGUAAAUUU SEQ ID NO. 14

Examples of other antisense based approaches for inhibiting Foxo1 expression is provided in U.S. Pat. No. 7,229,976.

The invention is illustrated herein by the experiments described above and by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

1. A method for treating or preventing a disease or disorder in a subject associated with impaired pancreatic function, comprising co-administering to the subject a therapeutically effective amount of a Foxo1 inhibitor and a therapeutically effective amount of a Notch inhibitor or Rock inhibitor, or both.

2. The method of claim 1, wherein the disease or disorder is selected from the group consisting of diabetes type 1, diabetic type 2, metabolic syndrome, glucose intolerance, hyperglycemia; decreased insulin sensitivity, increased fasting glucose, increased post-prandial glucose and obesity.

3. The method of claim 2, wherein the therapeutically effective amount is an amount that produces one or more effects selected from the group consisting of an increase in glucose tolerance, an increase in serum insulin, an increase insulin sensitivity, a decrease in fasting glucose, a decrease in post-prandial glucose, a decrease in weight gain, a decrease in fat mass, an increase in weight loss and the generation of gut ins+ cells.

4. The method of any of claim 1, wherein the Foxo1 inhibitor, Notch inhibitor or Rock inhibitor is administered to the gastrointestinal tract.

5. The method of any claim 1, wherein co-administering comprises (i) administering a dose of a Foxo1 inhibitor contemporaneous to a dose of a Notch inhibitor; and (ii) subsequent to step (i), administering one or more sequential doses of a Foxo1 inhibitor.

6. The method of claim 5, wherein administering a dose of a Foxo1 inhibitor contemporaneous to a dose of a Notch inhibitor comprises administering the Foxo1 inhibitor and Notch inhibitor within 12 hours of each other.

7. The method of claim 5, wherein administering one or more sequential doses of a Foxo1 inhibitor comprises administering at least one dose of a Foxo1 inhibitor at least once a day for at least three days.

8. The method of claim 7, wherein administering at least one dose of a Foxo1 inhibitor at least once a day for at least three days comprises administering 2 or more doses of a Foxo1 inhibitor a day for at least three successive days.

9. The method of claim 1, wherein the Foxo1 inhibitor or Notch inhibitor is administered in an enteric form so as to release the Foxo1 inhibitor or Notch inhibitor, or both, at a gut region comprising Ins− gut cells, or locally administered directly into or onto the gut region.

10. The method of claim 1, wherein a therapeutically effective amount of a Foxo1 inhibitor is co-administered with a therapeutically effective amount of a Rock inhibitor.

11. The method of claim 10, wherein the Foxo1 inhibitor or ROCK inhibitor is administered in an orally administrable enteric form so as to release the Foxo1 inhibitor or ROCK inhibitor, or both, at a gut region comprising Ins− gut cells, or locally administered directly into or onto the gut region.

12. The method of claim 2, wherein the therapeutically effective amount is an amount that generates gut ins+ cells in the subject.

13. A pharmaceutical composition for treating or preventing a disease or disorder in a subject associated with impaired pancreatic function, comprising an effective amount of a Foxo1 inhibitor and a Notch inhibitor or ROCK inhibitor, or both.

14. The pharmaceutical composition of claim 13, wherein the effective amount is an amount that produce an effect selected from the group consisting of an increase in glucose tolerance, an increase in serum insulin, an increase insulin sensitivity, a decrease in fasting glucose, a decrease in post-prandial glucose, a decrease in weight gain, a decrease in fat mass, an increase in weight loss and the generation gut Ins+ cells.

15. The pharmaceutical composition of claim 13 comprising a Foxo1 inhibitor and a Notch inhibitor that is in an orally administrable enteric form so as to release the Foxo1 inhibitor or Notch inhibitor or both at a gut region comprising gut ins− cells, or is in a form for local administration onto or into the gut region.

16. The pharmaceutical composition of claim 13 comprising a Foxo1 inhibitor and a ROCK inhibitor that is in an orally administrable enteric form so as to release the Foxo1 inhibitor or Notch inhibitor or both at a gut region comprising gut ins− cells or is in a form for local administration onto or into the gut region.

17. The pharmaceutical composition of claim 13, wherein the Notch inhibitor is selected from the group consisting of DBZ, MK-0752, PF-03084014, and LY450139.

18. The pharmaceutical composition of claim 13, wherein the ROCK inhibitor is selected from the group consisting of Y-27632, H-1152, and Wf-536.

19. The pharmaceutical composition of claim 13, wherein the Foxo1 inhibitor is selected from the group consisting of FBT9 and FBT10.

20. A method for producing enteroendocrine cells that make and secrete insulin in a subject, comprising co-administering to the subject an effective amount of a Foxo1 inhibitor and an effective amount of a Notch inhibitor or Rock inhibitor, or both.

21. The method of claim 20, wherein the insulin-producing enteroendocrine cells further produce one or more pancreatic hormones selected from the group consisting of glucokinase, and glut2 in response to administration of the agent.

22. The method of claim 20, wherein co-administering comprises (i) administering a dose of a Foxo1 inhibitor contemporaneous to a dose of a Notch inhibitor; and (ii) subsequent to step (i), administering one or more sequential doses of a Foxo1 inhibitor.

23. The method of claim 20, wherein the Foxo1 inhibitor or Notch inhibitor is administered in an enteric form so as to release the Foxo1 inhibitor or Notch inhibitor, or both, at a gut region comprising Ins− gut cells, or locally administered directly into or onto the gut region.

24. The method of claim 1, wherein the Notch inhibitor is selected from the group consisting of DBZ, MK-0752, PF-03084014, and LY450139.

25. The method of claim 1, wherein the ROCK inhibitor is selected from the group consisting of Y-27632, H-1152, and Wf-536.

26. The method of claim 1, wherein the Foxo1 inhibitor is selected from the group consisting of FBT9 and FBT10.


SEQUENCE LISTING (14 sequences)

SEQ ID NO: 1 - DNA, 21 bp, Artificial Sequence
  Synthetic short-hairpin RNA
gcaccgactttatgagcaacc

SEQ ID NO: 2 - DNA, 17 bp, Artificial Sequence
  Synthetic FOXO1-antisense
ttgggtcaggcggttca

SEQ ID NO: 3 - DNA, 20 bp, Artificial Sequence
  Synthetic FOXO3a-sense
cccagcctaaccagggaagt

SEQ ID NO: 4 - DNA, 16 bp, Artificial Sequence
  Synthetic FOXO3a-antisense
agcgccctgggtttgg

SEQ ID NO: 5 - DNA, 21 bp, Artificial Sequence
  Synthetic FOXO4-sense
cctgcacagcaagttcatcaa

SEQ ID NO: 6 - DNA, 21 bp, Artificial Sequence
  Synthetic FOXO4-antisense
ttcagcatccaccaagagctt

SEQ ID NO: 7 - RNA, 19 nt, Artificial Sequence
  Synthetic Target Sequence
cuauuauuguacaugauug

SEQ ID NO: 8 - RNA, 18 nt, Artificial Sequence
  Synthetic Target Sequence
cgaugauaccugauaaug

SEQ ID NO: 9 - RNA, 19 nt, Artificial Sequence
  Synthetic Target Sequence
ucguaaaccauuguaauua

SEQ ID NO: 10 - RNA, 19 nt, Artificial Sequence
  Synthetic Target Sequence
ccaggauaauugguuuuac

SEQ ID NO: 11 - RNA, 19 nt, Artificial Sequence
  Synthetic Target Sequence
ggugucaggcuaagaguua

SEQ ID NO: 12 - RNA, 19 nt, Artificial Sequence
  Synthetic Target Sequence
guaaugaugggcccuaauu

SEQ ID NO: 13 - RNA, 19 nt, Artificial Sequence
  Synthetic Target Sequence
gcaaacggcuucggucaac

SEQ ID NO: 14 - RNA, 19 nt, Artificial Sequence
  Synthetic Target Sequence
ggacaacaacaguaaauuu